(19) United States
(12) Patent Application Publication
(10) Pub. No.: US 2026/0345268 A1
(43) Pub. Date: Nov. 5, 2026

(54) SYSTEMS INCLUDING A GRAPHICAL USER INTERFACE FOR A SURGICAL ROBOTIC SYSTEM

(71) Applicant: Vicarious Surgical Inc., Waltham, MA (US)

(72) Inventors: Jeffrey Bail, Watertown, MA (US); Tabitha A. Solomon, Arlington, MA (US); Maxim Antinori, Arlington, MA (US); Edward Pioli, Maynard, MA (US)

(21) Appl. No.: 19/120,703

(22) PCT Filed: Oct. 31, 2023
(86) PCT No.: PCT/US2023/036369

§ 371 Date: Apr. 11, 2025

Related U.S. Application Data
(60) Provisional application No. 63/421,100, filed on Oct. 31, 2022.

(51) Int. Cl. A61B 34/37; A61B 34/00; A61B 34/20; A61B 90/00

(52) U.S. Cl. CPC A61B 34/37; A61B 34/20; A61B 34/25; A61B 90/361; A61B 2034/2055; A61B 2034/254

(57) ABSTRACT

Systems for a robotic arms assembly are disclosed. The system receives an input from either robotic arms or hand controllers. The system renders a graphical representation of the input on a graphical user interface (GUI) to provide the user with a visual representation of at least an engagement state between hands of the user and the hand controllers or a spatial awareness of the robotic arms within the cavity. The system overlays the GUI on live video footage on the display. The system renders on the GUI a graphical user interface element indicating a first mode of the surgical robotic system. The system receives a mode change indicator from the hand controllers, and instructs the GUI to change the graphical user interface element indicating the first mode responsive to the mode change indicator. In response the surgical robotic system to exit the first mode and activate a second mode.

### RELATED APPLICATIONS

This application claims the benefit of the filing date, under 35 U.S.C. § 119(e), of U.S.

Provisional Application No. 63/421,100, filed on Oct. 31, 2022, the entire contents of which are incorporated herein by reference.

### BACKGROUND OF THE DISCLOSURE

Surgical robotic systems permit a user (also described herein as an “operator” or a “user”) to perform an operation using robotically-controlled instruments to perform tasks and functions during a procedure. A visualization system is often used in collaboration with the surgical robotic system to output images or video of a surgical procedure.

The visualization system outputs graphical user interfaces allowing the user to interact with the output images and video. However, existing graphical user interfaces do not provide the freedom and flexibility to the user as they are performing a procedure. For instance, certain graphical user interfaces do not provide an easy to use interface for the user to quickly and efficiently navigate through different menus to select certain instruments, and configure certain features or settings of the robotic surgical systems.

### SUMMARY

A surgical robotic system is presented. The surgical robotic system includes a camera assembly, a display, a robotic arm assembly having robotic arms, and hand controllers graspable by a user of the surgical robotic system to control the robotic arms and the camera assembly. The surgical robotic system also includes a memory storing one or more instructions, a processor configured to or programmed to read the one or more instructions stored in the memory. The processor is operationally coupled to the robotic arm assembly, the hand controllers and the camera assembly. The processor is configured to receive input from the hand controllers. The processor is further configured to render a graphical representation of the input on a graphical user interface (GUI) to provide the user with a visual representation of at least an engagement state between hands of the user and the hand controllers or a spatial awareness of the robotic arms within the cavity. The processor is further configured to overlay the GUI on live video footage on the display. The processor is further configured to render on the GUI, a graphical user interface element indicating a first mode of the surgical robotic system. The processor is further configured to receive a mode change indicator from the hand controllers. The processor is further configured to in response to the mode change indicator, instructing the GUI to change the graphical user interface element indicating the first mode, and causing the surgical robotic system to exit the first mode, and activate a second mode.

A method of controlling a surgical robotic system is presented. The method includes generating with a processor configured to or programmed to read one or more instructions stored in memory a graphical user interface (GUI) on a display of the surgical robotic system, the processor operationally coupled to hand controllers graspable by a user of the surgical robotic system, a robotic arm assembly having robotic arms, and a camera assembly. The method further includes displaying on the display live video footage captured by the camera assembly, and overlaying the GUI on the live video footage on the display. The method further includes receiving by the processor input from one or more buttons or touch inputs on the hand controllers to control the robotic arm assembly. The method further includes rendering a graphical representation of the input on the GUI to provide the user with a visual representation of at least an engagement state between hands of the user and the hand controllers or a spatial awareness of the robotic arms within a cavity to control the surgical robotic system. The method further includes rendering on the GUI a graphical user interface element indicating a first mode of the surgical robotic system. The method further includes receiving by the processor a mode change indicator from the hand controllers, and responsive to the mode change indicator instructing the GUI to change the graphical user interface element indicating the first mode. The method further includes causing the surgical robotic system to exit the first mode, and activate a second mode.

A non-transitory computer readable medium storing computer-executable instructions is presented, and a processor that executes the stored instructions is presented for controlling a surgical robotic system. The processor is configured to perform the operations of displaying on a display live video footage captured by the camera assembly, and overlaying the GUI on the live video footage on the display. The processor is further configured to perform the operations of receiving by the processor input from one or more buttons or touch inputs on the hand controllers to control the robotic arm assembly. The processor is further configured to perform the operations of rendering a graphical representation of the input on the GUI to provide the user with a visual representation of at least an engagement state between hands of the user and the hand controllers or a spatial awareness of the robotic arms within a cavity to control the surgical robotic system. The processor is further configured to perform the operations of rendering on the GUI a graphical user interface element indicating a first mode of the surgical robotic system. The processor is further configured to perform the operations of receiving by the processor a mode change indicator from the hand controllers, and responsive to the mode change indicator instructing the GUI to change the graphical user interface element indicating the first mode. The processor is further configured to perform the operations of causing the surgical robotic system to exit the first mode, and activate a second mode.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 schematically depicts an example surgical robotic system in accordance with some embodiments.

FIG. 2A is an example perspective view of a patient cart including a robotic support system coupled to a robotic subsystem of the surgical robotic system in accordance with some embodiments.

FIG. 2B is an example perspective view of an example operator console of a surgical robotic system of the present disclosure in accordance with some embodiments.

FIG. 3A schematically depicts an example side view of a surgical robotic system performing a surgery within an internal cavity of a subject in accordance with some embodiments.

FIG. 3B schematically depicts an example top view of the surgical robotic system performing the surgery within the internal cavity of the subject of FIG. 3A in accordance with some embodiments.

FIG. 4A is an example perspective view of a single robotic arm subsystem in accordance with some embodiments.

FIG. 4B is an example perspective side view of a single robotic arm of the single robotic arm subsystem of FIG. 4A in accordance with some embodiments.

FIG. 5 is an example perspective front view of a camera assembly and a robotic arm assembly in accordance with some embodiments.

FIG. 6A is an example perspective view of a left hand controller for use in an operator console of a surgical robotic system in accordance with some embodiments.

FIG. 6B is an example perspective view of a right hand controller for use in an operator console of a surgical robotic system in accordance with some embodiments.

FIG. 7A is an example perspective view of a left hand controller for use in an operator console of a surgical robotic system in accordance with some embodiments.

FIG. 7B is an example perspective view of a right hand controller for use in an operator console of a surgical robotic system in accordance with some embodiments.

FIG. 8A is an example perspective view of a left hand controller for use in an operator console of a surgical robotic system in accordance with some embodiments.

FIG. 8B is an example perspective view of a right hand controller for use in an operator console of a surgical robotic system in accordance with some embodiments.

FIG. 9 is an example graphical user interface of a robot pose view including a frustum view of a cavity of a patient and a pair of robotic arms of the surgical robotic system, a robot pose view, and a camera view, in accordance with some embodiments.

FIG. 10 is an example graphical user interface of an engagement mode including a frustum view of a cavity of a patient and a pair of robotic arms of the surgical robotic system, a robot pose view, and a camera view, in accordance with some embodiments.

FIG. 11 is an example graphical user interface including a frustum view of a cavity of a patient and a pair of robotic arms of the surgical robotic system, situational awareness camera view panels, and an interactive menu for toggling settings of the surgical robotic system in accordance with some embodiments.

FIG. 12 is an example graphical user interface of a top level view menu, which includes a frustum view of a cavity of a patient and a pair of robotic arms of the surgical robotic system, a robot pose view, and a camera view, in accordance with some embodiments.

FIG. 13 is an example graphical user interface including an interactive menu for adjusting a camera brightness setting of the surgical robotic system, a frustum view of a cavity of a patient and a pair of robotic arms of the surgical robotic system, a robot pose view, and a camera view, in accordance with some embodiments.

FIG. 14 is an example graphical user interface including an interactive menu for toggling a camera view, a frustum view of a cavity of a patient and a pair of robotic arms of the surgical robotic system, a robot pose view, and a camera view, in accordance with some embodiments.

FIG. 15 depicts an example graphical user interface including a pillar box associated with a camera mode of operation, a frustum view of a cavity of a patient, a pair of robotic arms of the surgical robotic system, a robot pose view, and a camera view, in accordance with some embodiments.

FIG. 16 depicts an example graphical user interface including a pillar box associated with a scan mode of operation, a frustum view of a cavity of a patient, a pair of robotic arms of the surgical robotic system, a robot pose view, and a camera view, in accordance with some embodiments.

FIG. 17 depicts an example graphical user interface including a pillar box associated with an instrument mode, a frustum view of a cavity of a patient, a pair of robotic arms of the surgical robotic system, a robot pose view, and a camera view, in accordance with some embodiments.

FIG. 18 depicts an example graphical user interface including a pillar box associated with a pivot mode, a frustum view of a cavity of a patient, a pair of robotic arms of the surgical robotic system, robot pose view, and a camera view, in accordance with some embodiments.

FIG. 19 depicts an example graphical user interface including a pillar box associated with a travel mode, a frustum view of a cavity of a patient, a pair of robotic arms of the surgical robotic system, a robot pose view, and a camera view, in accordance with some embodiments.

FIG. 20 depicts an example graphical user interface including a pillar box associated with an insertion mode, a frustum view of a cavity of a patient, a pair of robotic arms of the surgical robotic system, a robot pose view, and a camera view, in accordance with some embodiments.

FIG. 21A depicts an example frustum view of a cavity of a patient, a pair of robotic arms of the surgical robotic system, and a robot pose view in accordance with some embodiments.

FIG. 21B depicts an example frustum view of a cavity of a patient, a pair of robotic arms of the surgical robotic system, and a camera view in accordance with some embodiments.

FIG. 22 is an example graphical user interface including a frustum view of a cavity of a patient and a pair of robotic arms of the surgical robotic system, and an interactive menu for toggling settings of the surgical robotic system in accordance with some embodiments.

FIG. 23A depicts an example graphical user interface of a top level view menu, which includes a frustum view of a cavity of a patient and a pair of robotic arms of the surgical robotic system, and a robot pose view, in accordance with some embodiments.

FIG. 23B depicts an example graphical user interface of a top level view menu, which includes a frustum view of a cavity of a patient and a pair of robotic arms of the surgical robotic system, and a camera view, in accordance with some embodiments.

FIG. 24 is an example graphical user interface including an interactive menu for adjusting a camera brightness setting of the surgical robotic system, a frustum view of a cavity of a patient and a pair of robotic arms of the surgical robotic system, and a camera view, in accordance with some embodiments.

FIG. 25 is an example graphical user interface including an interactive menu for toggling a camera view, a frustum view of a cavity of a patient and a pair of robotic arms of the surgical robotic system, and a camera view, in accordance with some embodiments.

FIG. 26 schematically depicts a graphical user interface including a camera view portion displaying a view from a camera assembly and a menu.

FIG. 27 is an example flowchart corresponding to changing a mode of operation of the surgical robotic system, in accordance with some embodiment.

FIG. 28 schematically depicts an example computing module of the surgical robotic system in accordance with some embodiments.

### DETAILED DESCRIPTION

While various embodiments of the invention have been shown and described herein, it will be clear to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Although some example embodiments may be described herein or in documents incorporated by reference as employing a plurality of units to perform example processes, it is understood that example processes may also be performed by one or a plurality of modules. Additionally, it is understood that the term controller/controller may refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or controllers or multiple different types of controllers or controllers may be employed in performing one or more processes. In some embodiments, different controllers or controllers may be implemented in different portions of a surgical robotic systems.

Some embodiments disclosed herein are implemented on, employ, or are incorporated into a surgical robotic system that includes a camera assembly having at least three articulating degrees of freedom and two or more robotic arms each having at least six articulating degrees of freedom and an additional degree of freedom corresponding to the movement of an associated end-effector (e.g., grasper, manipulator, and the like). In some embodiments, the camera assembly when mounted within a subject (e.g., a patient) can be moved or rotated in a pitch or yaw direction about 180 degrees such that the camera assembly can view rearwardly back towards the insertion site. As such, the camera assembly and the robotic arms can view and operate dexterously forward (e.g., away from the insertion site), to each side, in an upward or downward direction, as well as in the rearward direction to view backwards towards the insertion site. The robotic arms and the camera assembly can also move in the roll, pitch and yaw directions.

The large number of degrees of freedom in some surgical robotic systems described herein, in comparison to some conventional surgical robotic systems, enables movements of a robotic arm assembly and orientations of a robotic arm assembly not possible with some conventional surgical robotic arms and enables movements of a camera of a robotic camera assembly not possible in cameras for some conventional robotic surgical systems. For example, many conventional surgical robotic systems having two robotic arms and fewer degrees of freedom per arm may not be able to change a position or an orientation of a virtual chest of the robotic arms assembly while keeping instrument tips of end effectors of the robotic arms stationary. As another example, cameras of many conventional surgical robotic systems may only have degrees of freedom associated with movement of a support for the camera extending through a trocar and may have no independent degrees of freedom for movement relative to the support.

Some embodiments described herein provide methods and systems employing multiple different control modes, which may be described as a plurality of control modes herein, for controlling a surgical robotic system before, during or after a robotic arms assembly of the surgical robotic system is disposed within an internal body cavity of a subject. In some embodiments, the robotic arms assembly includes at least two robotic arms, which may be described as a “robotic arm assembly” or “arm assembly” herein. In some embodiments, the robotic arms assembly also includes a camera assembly, which may be also be referred to as a “surgical camera assembly”, or “robotic camera assembly” herein. Each control mode uses sensed movement of one or more hand controllers, and may also use input from one or more foot pedals, to control the robotic arm assembly and/or the camera assembly. A control mode may be changed from a current control mode to a different selected control mode based on operator input (e.g., provided via one or more hand controllers and/or a foot pedal of the surgical robotic system). In different control modes, the same movements of the hand controllers may result in different motions of the surgical robotic assembly.

When describing the control modes, a reference, an orientation or a direction of view of a “camera assembly” or a “camera” is referring to an orientation or a direction of a component or group of components of the surgical robotic arms assembly that includes one or more cameras or other imaging devices that can collectively change orientation with respect to the robotic arm assembly and provide image data to be displayed. For example, in some embodiments, the one or more cameras or other imaging devices may all be disposed in a same housing whose orientation can be changed relative to a support (e.g., support tube or support shaft) for the camera assembly.

Some embodiments employ a plurality of control modes including an instrument control mode, which may also be referred to as an “instrument mode” herein, as well as one or more additional control modes.

In some embodiments, the additional control modes include a scan mode, which may also be referred to herein as a “scanning mode” or a “survey mode”. In the scan mode, the camera changes orientation to change a direction or an orientation of view in response to movement of one or both of the hand controllers.

In some embodiments, the additional control modes include a perspective mode, which may also be referred to as a “view mode”, a “camera control mode”, a “camera mode”, a “framing control mode”, or a “framing mode” herein. In the camera/view mode, the surgical robotic system can rotate the camera, can translate a virtual chest of the robotic assembly, can pivot the chest of the robotic arms assembly or perform any combination of the aforementioned to change an orientation of a direction of view and a perspective of the camera in response to movement of one or both hand controllers while automatically maintaining a position and an orientation of the instrument tip of each robotic arm stationary.

In some embodiments, the additional control modes include a travel control mode, which may also be referred to as a “travel mode” or an “autotrack mode” herein. In some embodiments, the travel mode is one of multiple tracking modes, in which the surgical robotic arms assembly automatically adjusts so that a view of the camera tracks a position at a midpoint between the instrument tips. In the travel mode, the virtual chest of the robotic arms assembly can be translated, the robotic arms and the virtual chest of the robotic arms assembly together can be translated, an orientation of the virtual chest can be changed, an orientation of the camera can be changed, or any combination of the aforementioned, to automatically center the camera view on the instrument tips as the instrument tips are moved in response to movement of one or both hand controllers.

In some embodiments, the additional control modes include a pivot control mode, which may also be referred to as a “pivot mode” herein. In the pivot mode, which is a tracking mode, the orientation of the robotic chest, the camera or both can be changed to automatically center the camera view on the midpoint between the instrument tips as the instrument tips are moved in response to movement of one or both hand controllers.

In some embodiments, the additional control modes include a translate control mode, which may also be referred to as a “translate mode” herein. In the translation mode, which is a tracking mode, the chest of the robotic arms assembly or the chest and the arms can be translated together to automatically center the camera view on the midpoint between the instrument tips while the instrument tips are moved in response to movement of one or both hand controllers.

In some embodiments, the pivot mode, the travel mode and the translate mode may all be referred to as “tracking modes” herein because the view of the camera tracks a midpoint between instrument tips of the robotic arms in these modes.

Some embodiments employ additional features for controlling the robotic assembly. For example, some embodiments enable individual control of an elbow bias or an elbow elevation of a right robotic arm and a left robotic arm. Some embodiments employ a graphical user interface that identifies a current control mode of the surgical robotic system. Some embodiments employ a menu feature in which a menu is displayed on the graphical user interface and one or more of the hand controllers can be used to traverse menu options and select menu options.

Some embodiments may be employed with a surgical robotic system. A system for robotic surgery may include a robotic subsystem. The robotic subsystem includes at least a portion, which may also be referred to herein as a robotic arms assembly herein, that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks may be referred to as a surgical robotic module, a surgical robotic module or a robotic arms assembly herein. The surgical robotic module can include multiple different submodules or parts that may be inserted into the trocar separately. The surgical robotic module, surgical robotic module or robotic arms assembly can include multiple separate robotic arms that are deployable within the patient along different or separate axes. These multiple separate robotic arms may be collectively referred to as a robotic arm assembly herein. Further, a surgical camera assembly can also be deployed along a separate axis. The surgical robotic module, surgical robotic module, or robotic arms assembly may also include the surgical camera assembly. Thus, the surgical robotic module, or robotic arms assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic module that forms part of the present invention can form part of a surgical robotic system that includes a user workstation that includes appropriate sensors and displays, and a robot support system (RSS) for interacting with and supporting the robotic subsystem of the present invention in some embodiments. The robotic subsystem includes a motor and a surgical robotic module that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic module can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic module, which includes the robotic arms and the camera assembly. The motor assembly can include gears, motors, drivetrains, electronics, and the like, for powering the components of the surgical robotic module.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Like numerical identifiers are used throughout the figures to refer to the same elements.

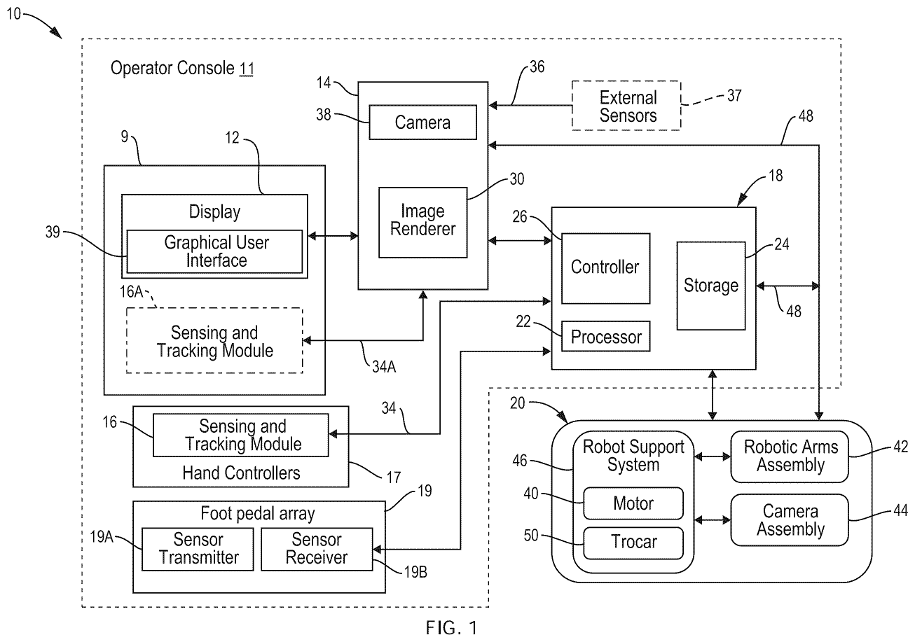

FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display 12, an image computing module 14, which may be a three-dimensional (3D) computing module, hand controllers 17 having a sensing and tracking module 16, and a computing module 18. Additionally, the operator console 11 may include a foot pedal array 19 including a plurality of pedals. The foot pedal array 19 may include a sensor transmitter 19A and a sensor receiver 19B. The image computing module 14 can include a camera 38. The camera 38, the controller 26 or the image renderer 30, or both, may render one or more images or one or more graphical user interface elements on the graphical user interface. For example, a pillar box associated with a mode of operating the surgical robotic system 10, or any of the various components of the surgical robotic system 10, can be rendered on the graphical user interface 39. Also live video footage captured by a camera assembly 44 can also be rendered by the controller 26 or the image renderer 30 on the graphical user interface 39.

The operator console 11 can include a visualization system 9 that includes a display 12 which may be any selected type of display for displaying information, images or video generated by the image computing module 14, the computing module 18, and/or the robotic subsystem 20. The display 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display 12 can also include an optional sensing and tracking module 16A. In some embodiments, the display 12 can include an image display for outputting an image from a camera assembly 44 of the robotic subsystem 20.

The hand controllers 17 are configured to sense a movement of the operator's hands and/or arms to manipulate the surgical robotic system 10. The hand controllers 17 can include the sensing and tracking module 16, circuity, and/or other hardware. The sensing and tracking module 16 can include one or more sensors or detectors that sense movements of the operator's hands. In some embodiments, the one or more sensors or detectors that sense movements of the operator's hands are disposed in the hand controllers 17 that are grasped by or engaged by hands of the operator. In some embodiments, the one or more sensors or detectors that sense movements of the operator's hands are coupled to the hands and/or arms of the operator. For example, the sensors of the sensing and tracking module 16 can be coupled to a region of the hand and/or the arm, such as the fingers, the wrist region, the elbow region, and/or the shoulder region. Additional sensors can also be coupled to a head and/or neck region of the operator in some embodiments. In some embodiments, the sensing and tracking module 16 can be external and coupled to the hand controllers 17 via electricity components and/or mounting hardware. In some embodiments, the optional sensor and tracking module 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking module 16 can employ sensors coupled to the torso of the operator or any other body part. In some embodiments, the sensing and tracking module 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking module 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing module 18 and hence employed by the surgical robotic system 10.

The sensors generate position and/or orientation data indicative of the position and/or orientation of the operator's hands and/or arms. The sensing and tracking modules 16 and/or 16A can be utilized to control movement (e.g., changing a position and/or an orientation) of the camera assembly 44 and robotic arms 42 of the robotic subsystem 20. The tracking and position data 34 generated by the sensing and tracking module 16 can be conveyed to the computing module 18 for processing by at least one processor 22.

The computing module 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arms 42 and/or the camera assembly 44. For example, the controller 26 can change a position and/or an orientation of at least a portion of the camera assembly 44, of at least a portion of the robotic arms 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arms 42, and the camera assembly 44.

The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms assembly 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arms assembly 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arms 42 and the cameras assembly 44 separately or together. The motor 40 can also provide mechanical power, electrical power, mechanical communication, and electrical communication to the robotic arms 42, the camera assembly 44, and/or other components of the RSS 46 and robotic subsystem 20. The motor 40 can be controlled by the computing module 18. The motor 40 can thus generate signals for controlling one or more motors that in turn can control and drive the robotic arms 42, including for example the position and orientation of each robot joint of each robotic arm, as well as the camera assembly 44. The motor 40 can further provide for a translational or linear degree of freedom that is first utilized to insert and remove each component of the robotic subsystem 20 through a trocar 50. The motor 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar 50 can be used to place at least a portion of the robotic subsystem 20 in an interior cavity of a subject (e.g., a patient) and can withdraw gas and/or fluid from a body cavity. The robotic subsystem 20 can be inserted through the trocar 50 to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking module 16, the robotic arms 42, the camera assembly 44, and the like), and for generating control signals in response thereto. The motor 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic arms assembly may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers 17 grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes an HMD, the display can include the built-in sensing and tracking module 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking module. In some embodiments, the sensing and tracking module 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensing and tracking module 16A for tracking at least a portion of the operator's head.

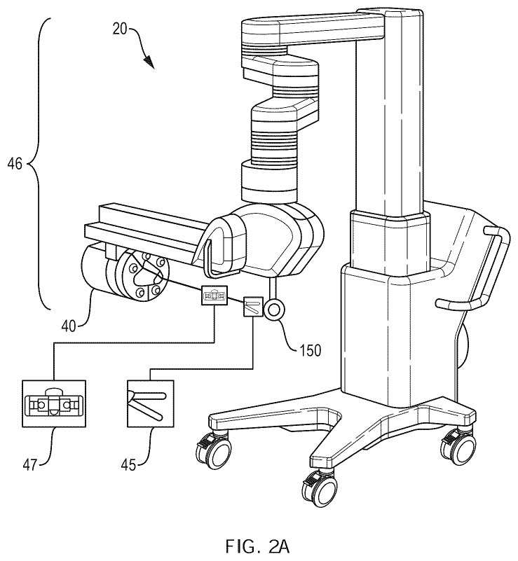

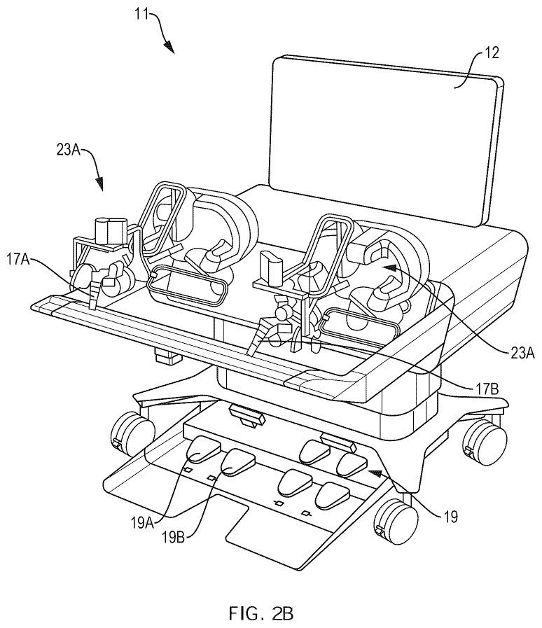

FIG. 2A depicts an example robotic arms assembly 20, which is also referred to herein as a robotic subsystem, of a surgical robotic system 10 incorporated into or mounted onto a mobile patient cart in accordance with some embodiments. In some embodiments, the robotic arms assembly 20 includes the RSS 46, which, in turn includes the motor 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes a display 12, hand controllers 17, and also includes one or more additional controllers, such as a foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A may releasably connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

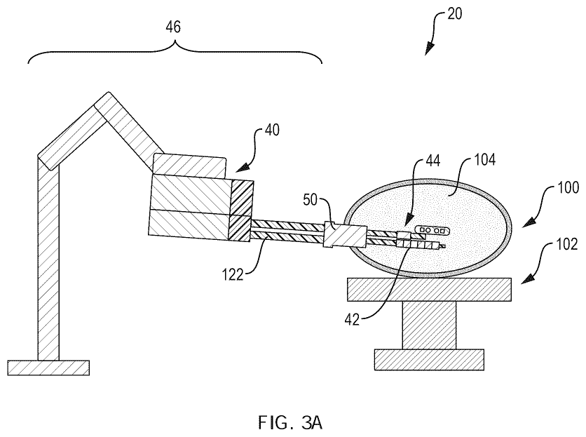

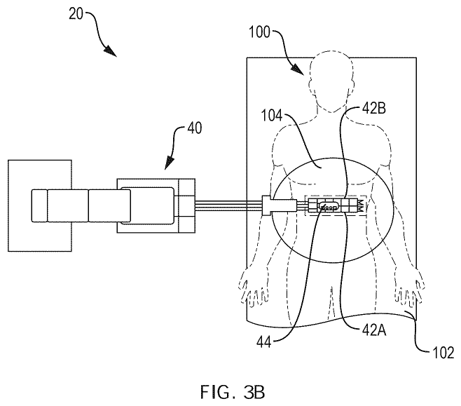

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic arms assembly 20 can be coupled to the motor 40 and at least a portion of the robotic arms assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robotic arm of the robotic arm assembly 42 and then followed by a second robotic arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

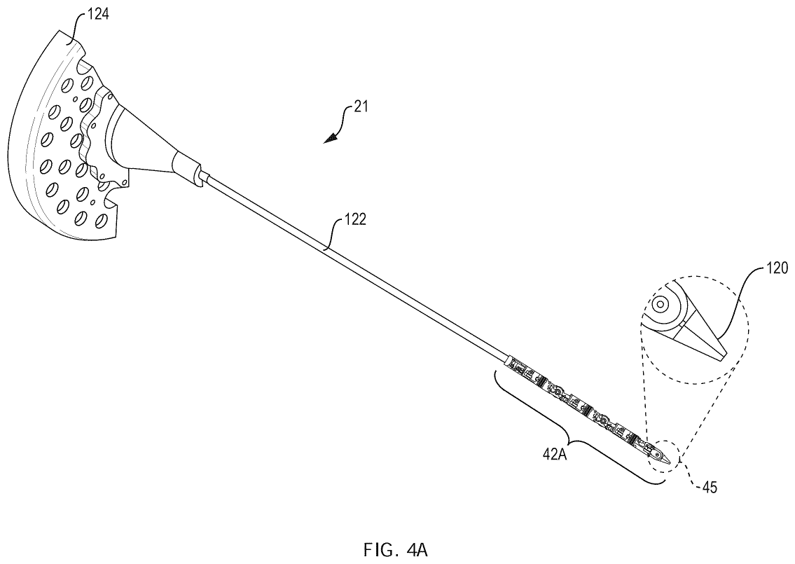

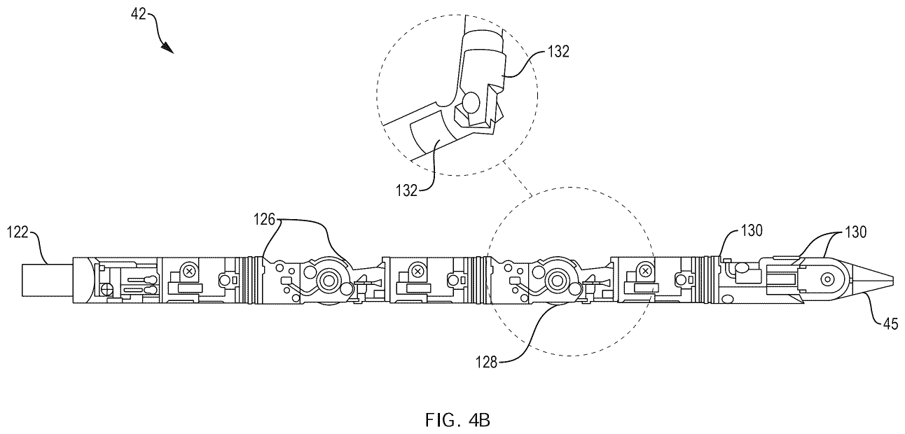

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensors 132 (e.g., capacitive proximity sensors), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

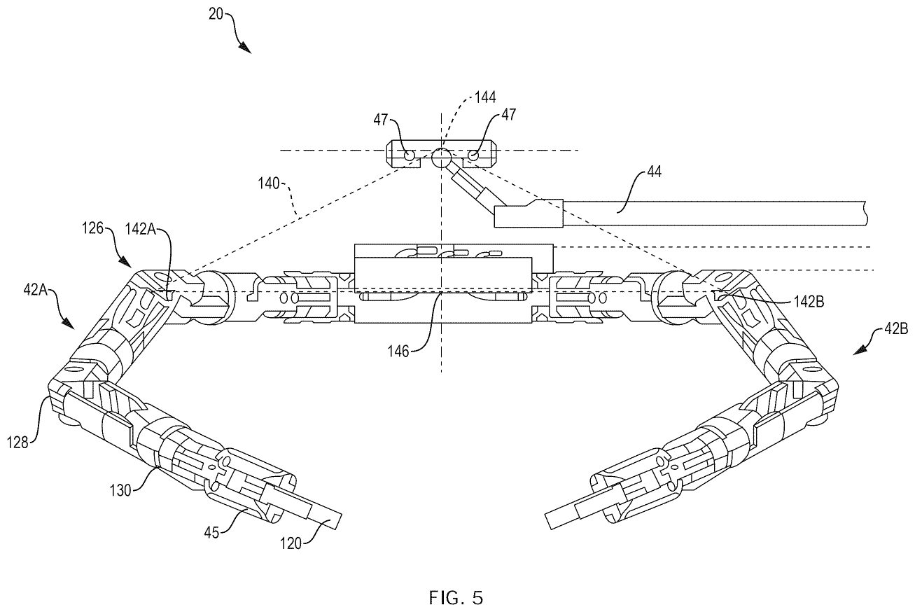

FIG. 5 illustrates a perspective front view of a portion of the robotic arms assembly 20 configured for insertion into an internal body cavity of a patient. The robotic arms assembly 20 includes a robotic arm 42A and a robotic arm 42B. The two robotic arms 42A and 42B can define, or at least partially define, a virtual chest 140 of the robotic arms assembly 20 in some embodiments. In some embodiments, the virtual chest 140 (depicted as a triangle with dotted lines) can be defined by a chest plane extending between a first pivot point 142A of a most proximal joint of the robotic arm 42A (e.g., a shoulder joint 126), a second pivot point 142B of a most proximal joint of the robotic arm 42B, and a camera imaging center point 144 of the camera(s) 47. A pivot center 146 of the virtual chest 140 lies in the middle of the virtual chest.

In some embodiments, sensors in one or both of the robotic arm 42A and the robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

Hand controllers for a surgical robotic system as described herein can be employed with any of the surgical robotic systems described above or any other suitable surgical robotic system. Further, some embodiments of hand controllers described herein may be employed with semi-robotic endoscopic surgical systems that are only robotic in part.

As explained above, controllers for a surgical robotic system may desirably feature sufficient inputs to provide control of the system, an ergonomic design and “natural” feel in use.

In some embodiments described herein, reference is made to a left hand controller and a corresponding left robotic arm, which may be a first robotic arm, and to a right hand controller and a corresponding right robotic arm, which may be a second robotic arm. In some embodiments, a robotic arm considered a left robotic arm and a robotic arm considered a right robotic arm may change due a configuration of the robotic arms and the camera assembly being adjusted such that the second robotic arm corresponds to a left robotic arm with respect to a view provided by the camera assembly and the first robotic arm corresponds to a right robotic arm with respect view provided by the camera assembly. In some embodiments, the surgical robotic system changes which robotic arm is identified as corresponding to the left hand controller and which robotic arm is identified as corresponding to the right hand controller during use. In some embodiments, at least one hand controller includes one or more operator input devices to provide one or more inputs for additional control of a robotic assembly. In some embodiments, the one or more operator input devices receive one or more operators inputs for at least one of: engaging a scanning mode, resetting a camera assembly orientation and position to a align a view of the camera assembly to the instrument tips and to the chest; displaying a menu, traversing a menu or highlighting options or items for selection and selecting an item or option, selecting and adjusting an elbow position, and engaging a clutch associated with an individual hand controller. In some embodiments, additional functions may be accessed via the menu, for example, selecting a level of a grasper force (e.g., high/low), selecting an insertion mode, an extraction mode, or an exchange mode, adjusting a focus, lighting, or a gain, camera cleaning, motion scaling, rotation of camera to enable looking down, etc.

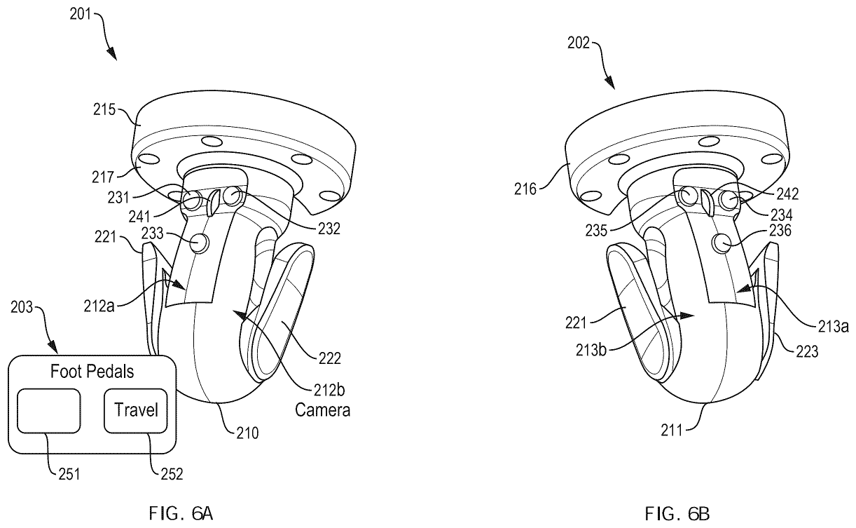

FIG. 6A depicts a left hand controller 201 and FIG. 6B depicts a right hand controller 202 in accordance with some embodiments. The left hand controller 201 and the right hand controller 202 each include a contoured housing 210, 211, respectively. Each contoured housing 210, 211, includes an upper surface 212a, 213a, an inside side surface 212b, 213 adjacent the upper surface, an outside side surface (not visible in these views) facing away from the inside side surface 212b, 213, and a lower surface (not visible in these views) facing away from the upper surface 212a, 213a.

In some embodiments, each hand controller 201, 202 includes a mounting assembly 215, 216, respectively. The mounting assembly 215, 216 may be used to attach, either directly or indirectly, the respective hand controller 201, 202 to a user console of a surgical robotic system. In some embodiments, the mounting assembly 215 defines holes 217, which may be countersunk holes, configured to receive a screw or bolt to connect the left hand controller 201 to a user console.

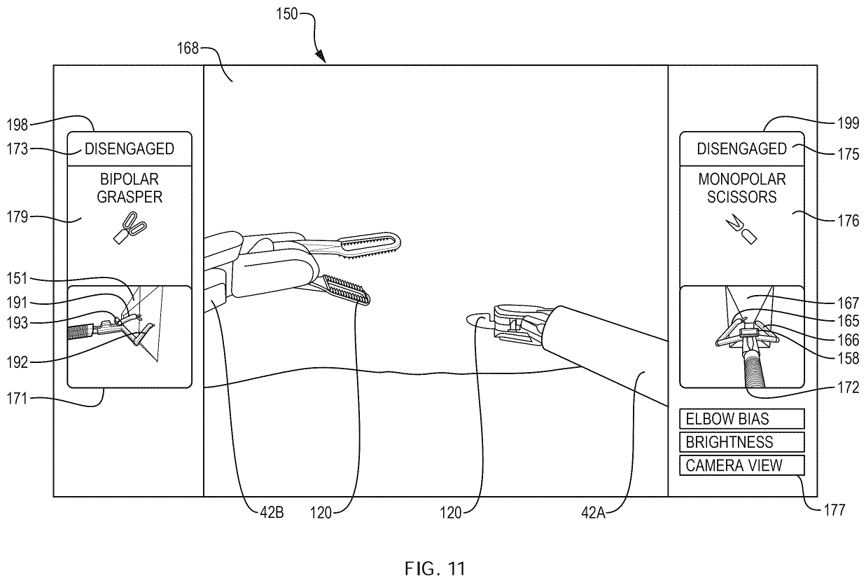

In some embodiments of the present disclosure, such as that depicted in FIGS. 6A and 6B, the hand controller includes two paddles, three buttons, and one touch input device. As will be explained herein, embodiments may feature other combinations of touch input devices, buttons, and levers, or a subset thereof. The embodiment shown as the left hand controller 201 features a first paddle 221 and a second paddle 222. Similarly, right hand controller 202 includes a first paddle 223 and a second paddle 224. In some embodiments, first paddle 221 is engaged with the second paddle 222 via one or more gears (not shown) so that a user depressing the first paddle 221 causes a reciprocal movement in the second paddle 222 and vice versa. Further description regarding a geared engagement between a first paddle and a second paddle is provided below with respect to FIGS. 11A and 11B. In another embodiment, first paddle 221 and second paddle 222 may be configured to operate independently. In embodiments employing reciprocal movement of the first and second paddle, a hand controller may employ only one signal indicating a deflection of the first lever and the second lever. In embodiments in which the first paddle and second paddle operate independently, a hand controller may employ a first signal indicating a deflection of the first paddle and a second signal indicating a deflection of the second paddle.

In some embodiments, the first paddle 221, 223 and the second paddle 222, 224 may be contoured to receive a thumb and/or finger of a user. In some embodiments, the first paddle 221, 223 extends from or extends beyond the outside side surface of the respective contoured housing 210, 211 the second paddle 222, 224 extends from or extends beyond the inside side surface 212b, 212c of the respective contoured housing. For each hand controller 210, 211, deflection or depression of the first paddle 221, 223, and the second paddle 222, 224, is configured to produce a signal that the surgical robotic system uses as an input to control a tool or an instrument tip (e.g., opening/closing an aperture of graspers/jaws of an instrument tip) at a distal end of a robotic arm of the surgical robotic system. For example, depressing first paddle and the second paddle may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

In some embodiments, a housing of a hand controller may be contoured. For example, in FIGS. 6A and 6B, the contoured housing 210, 211 includes a rounded shape. In some embodiments, a housing may be shaped to have a contour to match a contour of at least a portion of a thumb of a user's hand. In some embodiments, the contoured housing 210, 211, the first paddle 221, 223, and the second paddle 222, 224, may each be shaped to comfortably and ergonomically receive a respective hand of a user. In some embodiments, a housing of the hand controller, a lever or levers of a hand controller, buttons of a hand controller and/or one or more touch input devices may have shapes and/or positions on the hand controller for fitting different palm sizes and finger lengths.

Left hand controller 201 also includes a first button 231, a second button 232, and a third button 233. Similarly, right hand controller 202 also includes a first button 234, a second button 235 and a third button 236. As will be described herein, each button may provide one or more inputs that may be mapped to a variety of different functions of the surgical robotic device to control the surgical robotic system including a camera assembly and a robotic arm assembly. In an embodiment, input received via the first button 231 of the left hand controller 201 and input received via the first button 234 of the right hand controller 202 may control a clutch feature. For example, by engaging the first button 231, 234 a clutch is activated enabling movement of the respective left hand controller 201 or right hand controller 20, by the operator without causing any movement of a robotic arms assembly (e.g., a first robotic arm, a second robotic arm, and a camera assembly) of the surgical robotic system. When the clutch is activated for a hand controller, movement of the respective right hand controller or left hand controller is not translated to movement of the robotic assembly. In some embodiments, an operator engaging a hand controller input (e.g., tapping or pressing a button) activates the clutch and the operator engaging again (e.g., tapping or pressing the button again) turns off the clutch or exits a clutch mode. In some embodiments, an operator engaging a hand controller input (e.g., tapping or pressing a button and holding the button) activates the clutch and the clutch stays active for as long as the input is active and exits the clutch when the when the operator is no longer engaging the hand controller input (e.g., releasing the button). Activating the clutch or entering the clutch mode for a hand controller enables the operator to reposition the respective hand controller (e.g., re-position the left controller 201 within the range of motion of the left hand controller 201 and/or re-position the right hand controller 202 within a range of motion of the right hand controller 202) without causing movement of the robotic arms assembly itself.

The second button 232 of the left hand controller 201 may provide an input that controls a pivot function of the surgical robotic device. An operator engaging (e.g., pressing and holding) the second button 232 of the left hand controller 201 may engage a pivot function or a pivot mode that reorients the robotic arms assembly chest to center the camera on the midpoint between the instrument tips. The pivot function can be activated with a brief tap or held down to continuously track the instrument tips as they move, in accordance with some embodiments.

The second button 235 of the right hand controller 202 may provide input for entering a menu mode in which a menu is displayed on the graphical user interface 39 of the surgical robotic system and exiting a menu mode. The operator may activate a menu mode by pressing the second button 235 a first time and disengage the menu function by pressing the second button 235 a second time. The operator may be able to select options within the menu by navigating the menu using the left hand controller and/or the right hand controller when the menu mode is engaged. For example, the first touch input device 242 of the right hand controller 202 may be used to navigate the menu and to select a menu item in some embodiments. While in a menu mode, movement of the robotic in response to movement of the left hand controller 201 or the right hand controller 202 may be suspended. The menu mode and the selection of menu options are discussed in more detail below.

The third button 233 of the left hand controller and the third button of the right hand controller may provide an input that engages or disengages an instrument control mode of the surgical robotic system in some embodiments. A movement of at least one of the one or more hand controllers when in the instrument mode causes a corresponding movement in a corresponding robotic arm of the robotic assembly. The instrument control mode will be described in more detail below.

The left hand controller 201 further includes a touch input device 241. Similarly, the right hand controller 202 further includes a touch input device 242. In an embodiment, the touch input device 241, 242 may be a scroll wheel, as shown in FIGS. 6A and 6B. Other touch input devices that may be employed include, but are not limited to, rocker buttons, joy sticks, pointing sticks, touch pads, track balls, trackpoint nubs, etc.

The touch input device 241, 242 may be able to receive input through several different forms of engagement by the operator. For example, where the touch input device 241, 242 is a scroll wheel, the operator may be able to push or click the first touch input device 241, 242, scroll the first touch input device 241, 242 backward or forward, or both.

In some embodiments, scrolling the first touch input device 241 of the left hand controller 241 forward may activate a zoom in function to magnify a view provided by the camera assembly of the surgical robotic system and displayed to the operator, and scrolling backward with first touch input device 241 may provide a zoom out function to reduce the view provided by the camera assembly of the surgical robotic device and displayed to the operator, or vice versa. In embodiments, the zoom function may be mechanical or digital. In some embodiments, the zoom function may be mechanical in part and digital in part (e.g., a mechanical zoom over one zoom range, and a mechanical zoom plus a digital zoom over another zoom range).

In some embodiments, clicking or depressing first touch input device 241 may engage a scan mode of the surgical robotic system. When in a scan mode, a movement of at least one of the left hand controller 201 or the right hand controller 202 causes a corresponding change in an orientation of a camera assembly of the robotic arms assembly without changing a position or orientation of either robotic arm of the surgical robotic system. In another embodiment, pressing and holding the first touch input device 241 may activate the scan mode and releasing the first touch input device 241 may end the scan mode of the surgical robotic system. In some embodiments, releasing the scan mode returns the camera to the orientation it was in upon entering scan mode. In some embodiments, a function may be provided for locking the orientation upon exiting the scan mode (e.g., to change the “horizon” line).

In some embodiments, when in a menu mode and a left elbow menu item is selected, the first touch input device 241 of the left hand controller 201 may be used for selection of a direction and degree of left elbow bias. As used herein, elbow bias refers to the extent by which the virtual elbow of the robotic arm is above or below a neutral or default position.

In some embodiments, when in a menu mode, an operator may be able to select options within the menu by navigating the menu using the left hand controller and/or the right hand controller. For example, when in the menu mode, the touch input device 242 (e.g., scroll wheel) of the right hand controller provide a set of inputs for traversing a displayed menu and selecting an item in a displayed menu. For example, by scrolling forward on touch input device 242 the operator may move up the menu and by scrolling backwards with touch input device 242 the user may move down the menu, or vice versa. In an embodiment, by clicking first touch input device 242 the operator may make a selection within a menu. Use of the touch input device 242 and the menu mode are discussed in more detail below.

In some embodiments, the touch input device 242 of the right hand controller 202 may be used to control right elbow bias when a right elbow bias menu item has been selected.

Functions of various buttons and the touch input device described above with respect to the left hand controller above may instead be assigned to the right hand controller, and functions of various buttons and the touch input device described above with respect to the right hand controller may instead be assigned to the left hand controller in some embodiments.

FIG. 6A also shows a schematic depiction 203 of a first foot pedal 251 and second foot pedal 252 for receiving operator input. As shown in FIG. 6A, in some embodiments the first foot pedal 251 engages a camera control mode, also described herein as a view control mode, an image framing control mode, or a camera framing control mode of the surgical robotic system and the second foot pedal 252 engages a travel control mode of the surgical robotic system.

In some embodiment, when the camera control mode is activated e.g., using the foot pedal 251, movement of the left hand controller 201 and/or the right hand controller 202 by the operator may provide input that is interpreted by the system to control a movement of and an orientation of a camera assembly of the surgical robotic system while keeping positions of instrument tips of robotic arms of the robotic arms assembly constant.

In some embodiments, when the travel control mode is activated e.g., using the foot pedal 252, the left hand controller 201 and the right hand controller 202 may be used to move the robotic arm assembly of the surgical robotic system in a manner in which distal tips of the robotic arms direct or lead movement of a chest of the robotic arms assembly through an internal body cavity. In the travel control mode, a position and orientation of the camera assembly, of the chest, or of both is automatically adjusted to maintain the view of the camera assembly directed at the tips (e.g., at a point between a tip or tips of a distal end of the first robotic arm and a tip or tips of a distal end of the second robotic arm). This may be described as the camera assembly being pinned to the chest of the robotic arms assembly and automatically following the tips. Further detail regarding the travel control mode is provided below.

In different embodiments, different functions may map to different buttons and different touch input devices of the hand controllers. In different embodiments, different or other functions may correspond to buttons and touch input devices of the hand controllers that have a same physical structure.

For example, in some embodiments, compared with FIGS. 6A, 6B, the first button 231, 234 can provide an input that engages or disengages an instrument control mode of the surgical robotic system. In some embodiments, the hand controller 201, 202 may not include an engage/disengage button. Instead, an operator may put his/her head close to a display such that his/her head is within a certain distance of the display and detected by a sensor, and the operator can squeeze the paddles 221-224 to engage/disengage. In some embodiments, compared with FIGS. 6A, 6B, the second button 232 can engage a camera control mode. When the camera control mode is activated, movement of the left hand controller 201 and/or the right hand controller 202 by the operator may provide input that is interpreted by the system to control a movement of and an orientation of a camera assembly of the surgical robotic system while keeping positions of instrument tips of robotic arms of the robotic assembly constant. In some embodiments, the first pedal 251 can engage a pivot mode. In some embodiments, the functionality of the pivot mode may be consolidated into the camera mode and the system does not need to have a pivot mode. In some embodiments, compared with FIGS. 6A, 6B, the first pedal 252 can engage a translate mode.

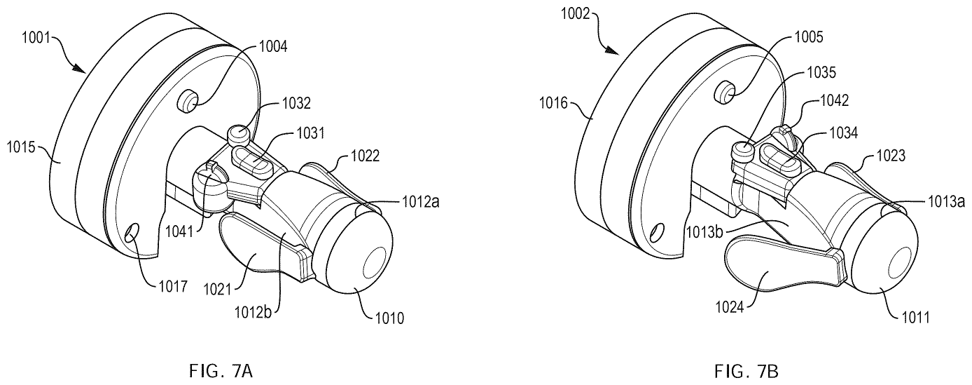

FIGS. 7A and 7B depict another embodiment according to the present disclosure featuring a left hand controller 1001 and a right hand controller 1002. The left hand controller 1001 includes a contoured housing 1010, and the right hand controller 1002 includes a contoured housing 1011. Each contoured housing 1010, 1011, includes an upper surface 1012a, 1013a, an inside side surface 1012b, 1013b adjacent the upper surface, an outside side surface (not visible in these views) facing away from the inside side surface 1012b, 1013b, and a lower surface (not visible in these views) facing away from the upper surface 1012a, 1013a.

Each hand controller 1001, 1002 includes a mounting assembly 1015, 1016, respectively. The mounting assembly 1015, 1016 may be used to attach, either directly or indirectly, each of the respective hand controllers 1001, 1002 to a surgeon console of a surgical robotic system. The mounting assembly 1015 includes an aperture 1017 and the mounting assembly 1016 defines an aperture 1018. The apertures 1017, 1018 may be countersunk apertures, configured to receive a screw or bolt to connect the respective hand controller 1001, 1002 to a surgeon console. The mounting assembly 1015 includes a button 1004 and the mounting assembly 1016 includes a button 1005. The buttons 1004, 1005 provide an input to toggle between insertion and extraction of one or more robotic arm assemblies 42A, 42B as well as the camera assembly 44. For example, the button 1004 can be used to insert or extract a first robotic arm 42A and the button 1005 can be used to insert or extract a second robotic arm 42B.

Each of the left hand controller 1001 and the right hand controller 1002 also includes a first button 1031, 1034, a second button 1032, 1035, a touch input device 1041, 1042 (e.g., a joy stick, or scroll wheel), respectively. In each hand controller 1001, 1002, the first button 1021, 1034, the second button 1032, 1035, and the touch input device 1041, 1042 are disposed on or at an upper surface 1012a, 1013a of the housing 1010, 1011, respectively. In some embodiments, the first button 1021, 1034, the second button 1032, 1035, and the touch input device 1041, 1042 are disposed on or at a portion of the upper surface 1012a, 1013a that projects from the upper surface. For each hand controller 1001, 1002, a lever (not visible in this view) extends from the respective outside side surface (not visible in this view). In some embodiments, a different mechanism may be used for a grasping input on a hand controller. For example, in some embodiments a hand controller may include a least one “pistol trigger” type button that can be pulled back to close and released to open instead of or in addition to a lever or levers.

The left hand controller 1001 includes a first paddle 1021 and a second paddle 1022. Similarly, right hand controller 1002 includes a first paddle 1023 and a second paddle 1024. In some embodiments, the first paddle 1021, 1023 is engaged with the second paddle 1022, 1024 of each hand controller 1001, 1002 via one or more gears (not shown) so that a user depressing the first paddle 1021, 1023 causes a reciprocal movement in the second paddle 1022, 1024 and vice versa, respectively. In another embodiment, the first paddle 1021, 1023 and the second paddle 1022, 1024 of each hand controller may be configured to operate independently. In embodiments employing reciprocal movement of the first and second paddles, the hand controller 1001, 1002 may employ some form of a signal or other indicator indicating a deflection of the first paddle 1021, 1023 and the second paddle 1022, 1024. In embodiments in which the first paddle and second paddle operate independently, the hand controller 1001, 1002 may employ a first signal or other indicator indicating a deflection of the first paddle 1021, 1023 and a second signal or other indicator indicating a deflection of the second paddle 1022, 1024.

In some embodiments, the first paddle 1021, 1023 and the second paddle 1022, 1024 may be contoured to receive a thumb and/or finger of a user. In some embodiments, the first paddle 1021, 1023 extends from or extends beyond the outside side surface of the respective contoured housing 1010, 1011 the second paddle 1022, 1024 extends from or extends beyond the inside side surface 1012b, 1013b of the respective contoured housing. For each hand controller 1010, 1011, deflection or depression of the first paddle 1021, 1023, and the second paddle 1022, 1024, is configured to trigger a signal that the surgical robotic system uses as an input to control a tool or an instrument tip (e.g., opening/closing an aperture of graspers/jaws of an instrument tip) at a distal end of a robotic arm of the surgical robotic system. For example, depressing the first paddle 1021, 1023 and the second paddle 1022, 1024 may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

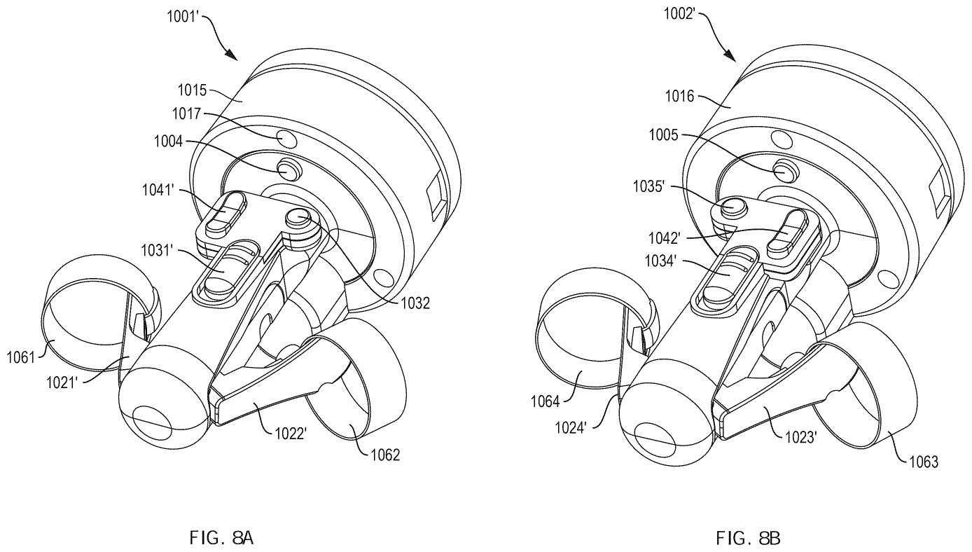

In some embodiments, each of the first paddle 1021, 1023 and the second paddle 1022, 1024 can have a loop to receive a thumb and/or finger of a user, as further described with respect to FIGS. 8A and 8B. In some embodiments, parameters (e.g., length, angle, finger ergonomics, and the like) of each of the first paddle 1021, 1023 and the second paddle 1022, 1024 can be adjusted.

The contoured housing 1010, 1011 may be configured to comfortably and ergonomically mate with a corresponding hand of the operator. The operator may engage with the respective hand controller 1001, 1002 by placing the thumb of the respective hand on the second paddle 1022, 1024, positioning the pointer finger or middle finger of the respective hand on or over the projecting portion of the upper surface 1013a, 1013a on which the first button 1021, 1034, the second button 1032, 1035, and the touch input device 1041, 1042 are disposed, and by positioning at least, the middle finger or ring finger of the respective hand on or over the first paddle 1021, 1024.

Although various example embodiments described herein assign certain functions to certain buttons and to certain touch input devices, one of ordinary skill of the art in view of the present disclosure will appreciate that which functions are ascribed to which buttons and touch input devices may be different in different embodiments. Further, one of ordinary skill of the art in view of the present disclosure will appreciate that additional functions not explicitly described herein may be assigned to some buttons and some touch input devices in some embodiments. In some embodiments, one or more functions may be assigned to a foot pedal of a surgical robotic system that includes one or more hand controllers as described herein.

By way of example, a set of functions that may be controlled by the left hand controller 1001 and the right hand controller 1002 for some embodiments of the present technology will now be described.

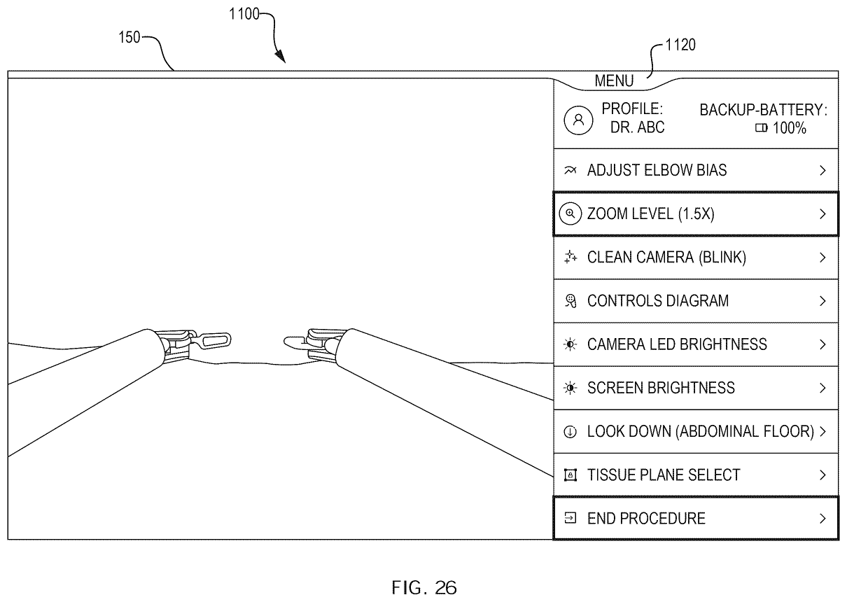

For the left hand controller 1001, pressing or pressing and holding the first button 1004 may trigger a signal used to engage an insertion or extraction for a left robotic arm assembly and/or a camera assembly of the surgical robotic system. Pressing or pressing and holding the first button 1031 may trigger a signal used to control a clutch function for the left hand controller of the surgical robotic system. Pressing or pressing and holding the second button 1032 may trigger a signal used to engage or disengage a camera control mode of the surgical robotic system. Scrolling the touch input device 1041 forward may activate a zoom in function to magnify a view provided by the camera assembly of the surgical robotic system and displayed to the operator, and scrolling backward with first touch input device 1041 may provide a zoom out function to reduce the view provided by the camera assembly of the surgical robotic device and displayed to the operator, or vice versa. Scrolling the touch input device 1041 may trigger a signal used to select left elbow bias when an elbow bias function is activated using a menu 1120 (as illustrated in FIG. 26).

For the right hand controller 1002, pressing or pressing and holding the first button 1005 may trigger a signal used to engage an insertion or extraction for a right robotic arm assembly and/or a camera assembly of the surgical robotic system. Pressing or pressing and holding the first button 1034 may trigger a signal used to control a clutch function for the right hand controller of the surgical robotic system. Clicking or depressing the second button 1035 may engage a scan mode of the surgical robotic system. When in a scan mode, a movement of at least one of the left hand controller 1001 or the right hand controller 1002 causes a corresponding change in an orientation of a camera assembly of the robotic assembly without changing a position or orientation of either robotic arm of the surgical robotic system. In another embodiment, pressing and holding the second button 1035 may activate the scan mode and releasing the second button 1035 may end the scan mode of the surgical robotic system. In some embodiments, releasing the scan mode returns the camera to the orientation it was in upon entering the scan mode. In some embodiments, a function may be provided for locking the orientation upon exiting the scan mode (e.g., to change the “horizon” line).

FIGS. 8A and 8B depict another embodiment according to the present disclosure featuring a left hand controller 1001′ and a right hand controller 1002′. Compared with the hand controllers 1001, 1002 in FIGS. 7A and 7B, some buttons of the hand controllers 1001′, 1002′ have the same button type but different functions. For example, the second button 1035′ of the right hand controller 1002′ may trigger a signal used to turn on or turn off a menu. Compared with the hand controllers 1001, 1002 in FIGS. 7A and 7B, some buttons of the hand controllers 1001′, 1002′ may have a different button type and/or different functions. For example, touch input device 1041′ for the left hand controller 1001′ may have a three-way switch button type. Switching or holding the touch input device 1041′ to the center may trigger a signal used to engage or disengage a scan mode of the surgical robotic system.

Switching the touch input device 1041′ forward may activate a zoom in function to magnify a view provided by the camera assembly of the surgical robotic system and displayed to the operator, and switching backward with first touch input device 1041′ may provide a zoom out function to reduce the view provided by the camera assembly of the surgical robotic device and displayed to the operator, or vice versa. Switching the touch input device 1035′ upward may trigger a signal used to traverse a menu when the menu is displayed or a menu mode is active. Touch input device 1042′ for the right hand controller 1002′ may have a three-way switch button type. Switching the touch input device 1042′ may trigger a signal used to traverse a menu or highlight a portion of the menu when the menu is displayed or a menu mode is active by pressing the touch input device 1035′. Switching forward on touch input device 1042′ may move up the menu and switching backwards with touch input device 1042′ may move down the menu, or vice versa. Clicking first touch input device 1042′ may trigger a signal used to select a highlighted portion or of the menu or feature on the menu when the menu is displayed. In some embodiments, switching the touch input device 1042′ may trigger a signal used to select right elbow bias when the elbow bias function is activated using the menu. Compared with the hand controllers 1001, 1002 in FIGS. 7A and 7B, the hand controllers 1001′, 1002′ may have the first paddles 1021′, 1023′ and second paddles 1022′, 1024′ to couple to finger loops 1061, 1062, 1063, 1064, respectively. Each finger loop can be a Velcro type. In some embodiments (not illustrated), each finger loop can be a hook type. Deflection or depression of the first paddle 1021′, 1023′, and the second paddle 1022′, 1024′, is configured to trigger a signal to control a tool or an instrument tip (e.g., opening/closing an aperture of graspers/jaws of an instrument tip) at a distal end of a robotic arm of the surgical robotic system. For example, depressing first paddle 1021′, 1023′ and the second paddle 1022′, 1024′ may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate). Compared with the hand controllers 1001, 1002 in FIGS. 7A and 7B, first buttons 1031′, 1034′ may have a slider button type. Sliding the first button 1031′, 1034′ may trigger a signal used to control a clutch function for the corresponding hand controller of the surgical robotic system.

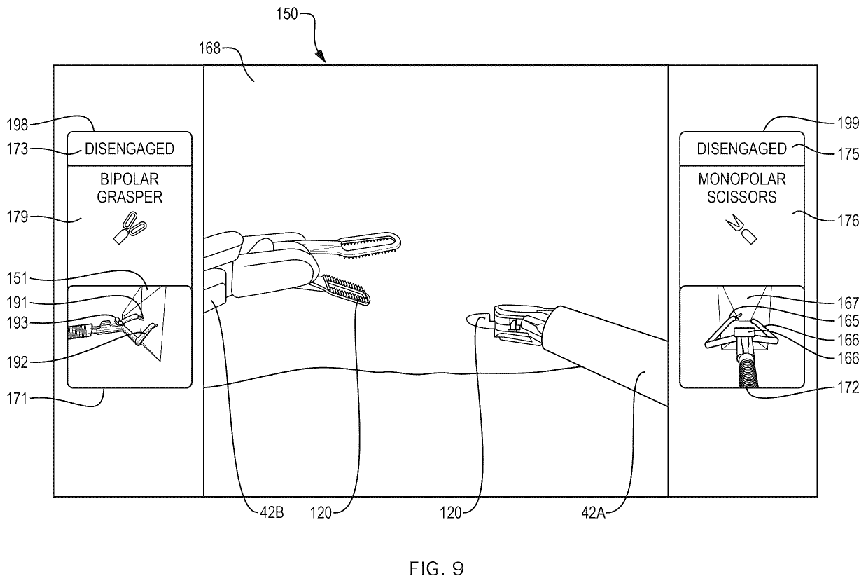

FIG. 9 is a graphical user interface 150 that is formatted to include a left pillar box 198 and a right pillar box 199 to the left and right of a live video footage 168, respectively, of a cavity of a patient. The graphical user interface 150 can be overlaid over the live video footage 168. In some embodiments, the live video footage 168 is formatted by the controller 26 to accommodate the left pillar box 198 and the right pillar box 199. In some embodiments, the live video footage 168 can be displayed on display 12, with a predetermined size and location on the display 12, and the left pillar box 198 and the right pillar box 199 can be displayed on either side of the live video footage 168 with a certain size based on the remaining area on the display 12 that is not occupied by the live video footage 168. The graphical user interface 150 includes multiple different graphical user interface elements, which are described below in more detail.

Robotic arms 42B and 42A are also visible in the live video footage. The left pillar box 198 can include a status identifier 173, for example, an engaged or disengaged status identifier associated with an instrument tip 120 of the robotic arm 42B. The “engaged” status identifier 173 indicates that the user's left hand and arm are engaged with the left hand controller 201 and therefore the instrument tip 120 is also engaged. The “disengaged” status identifier 173 indicates that the user's left hand and arm are not engaged with the hand controller 201 and therefore the instrument tip 120 is also disengaged. When the user's left hand and arm are disengaged with the left hand controller 201, the surgical robotic system 10 can be completely disengaged. That is the surgical robotic system 10 can remain on, but it is unresponsive until the user's hands reengage with the hand controllers. The instrument tip 120 can be represented by iconographic symbol 179 that includes a name of the instrument tip 120 to provide confirmation to the user of what type of end effector or instrument tip is currently in use. In FIG. 9, the instrument tip 120 represented by the iconographic symbol 179 is a bipolar grasper. Notably, the present disclosure is not limited to the bipolar grasper or scissor shown in FIG. 9.

Similarly, the right pillar box 199 can include a status identifier 175 associated with an instrument tip 120 of the robotic arm 42A for example, engaged or disengaged status identifier. In some embodiments, based on the status of the end effector, the graphical user interface can also provide a visual representation of the status in addition to text. For example, the end effector iconography can be “grayed out” or made less prominent if it is not disengaged.

The status identifier 175 can be “engaged” thereby indicating that the user's right hand and arm are engaged with the right hand controller 202 and therefore the instrument tip 120 is also engaged. Alternatively, the status identifier 175 can be “disengaged” thereby indicating that the user's right hand and arm are not engaged with the right hand controller 202 and therefore the instrument tip 120 is also disengaged. The instrument tip 120 can be represented by iconographic symbol 176 that includes a name of the instrument tip 120 to provide confirmation to the user of what type of end effector or instrument tip is currently in use. In FIG. 9, the instrument tip 120 represented by iconographic symbol 176 is a monopolar scissors. Notably, the present disclosure is not limited to the monopolar scissors shown in FIG. 9.

The left pillar box 198 can also include a robot pose view 171. The robot pose view 171 includes a simulated view of the robotic arms 42B and 42A, the camera assembly 44, and the support arm thereby allowing the user to get a third person view of the robotic arm assembly 42, the camera assembly 44, and the robot support system 46. The simulated view of the robotic arms 42B and 42A represented by a pair of simulated robotic arms 191 and 192. The simulated view of the camera assembly 44 is represented by a simulated camera 193. The robot pose view 171 also includes a simulated camera view associated with a cavity, or a portion of the cavity, of a patient, which is representative of the placement, or location of the pair of robotic arms 191 and 192 relative to a frustum 151. More specifically the camera view can be the field of view of the camera assembly 44 and is equivalent to the frustum 151.

The right pillar box 199 can also include a robot pose view 172 that includes a simulated view of the robotic arms 42B and 42A, the camera assembly 44, the support arm thereby allowing the user to get a third person view of the robotic arm assembly 42, the camera assembly 44, and the support arm. The simulated view of the robotic arms 42B and 42A are a pair of simulated robotic arms 165 and 166. The simulated view of the camera assembly 44 is represented by a simulated camera 193. The robot pose view 172 also includes a simulated camera view associated with a cavity, or a portion of the cavity, of the patient, which is the placement, or location of the pair of robotic arms 165 and 166 relative to a frustum 167. More specifically, the camera view can be the camera's field of view which is the frustum 167. The robot pose view 172 provides elbow height awareness, and situational awareness especially with driving in up facing/flip facing configurations.

Situational awareness can be characterized as a way of understanding certain robotic elements with respect to time and space when the robotic arms 42A and 42B are inside the cavity of the patient. For example, as shown in the robot pose view 171 the elbow of the simulated robotic arm 192 is bent downwards thereby providing the user with the ability to know how the elbow of the actual robotic arm 42A is actually oriented and positioned within the cavity of the patient. It should be noted that because of the positioning of the camera assembly 44 with respect to the robotic arms 42A and 42B, the entire length of the robotic arms 42A and 42B may not be visible in the live video footage 168. As a result, the user may not have visualization of how the robotic arms 42A and 42B are oriented and positioned within the cavity of the patient. The simulated robotic arms 165 and 166, as well as simulated robotic arms 191 and 192 provide the user with the situational awareness of at least the position and orientation of the actual robotic arms 42A and 42B within the cavity of the patient.

There can be two separate views (the robot pose view 171 and the robot pose view 172) from two different viewpoints on each side of the graphical user interface 150 that is rendered on display 12. The robot pose view 171 and the robot pose view 172 automatically update to stay centered on the trocar 50 while maintaining the robotic arms 42A and 42B in view. The robot pose views 171 and 172 also provide the user with spatial awareness.

Spatial awareness can be characterized as the placement or position of the robotic arms 42A and 42B as viewed in robot pose views 171 and 172 relative to other objects in the cavity and the cavity itself. The robot pose views 171 and 172 provide the user with the ability to determine where the actual robotic arms 42A and 42B are located within the cavity by viewing the simulated robotic arms 191 and 192 in the robot pose view 171 and simulated robotic arms 165 and 166 in robot pose view 172. For example, the robot pose view 171 illustrates the position and location of the simulated robotic arms 191 and 192 relative to the frustum 151. The robot pose view 171 depicts the simulated robotic arms 191 and 192 with respect to the frustum 151, from a side view of the support arm and the simulated robotic arms 191 and 192 that are attached to the support arm. This particular robot pose provides the user with the ability to better ascertain proximity to anatomical features within the cavity.

The robot pose view 172 can also provide the user with the ability to better ascertain how close the actual robotic arms 42A and 42B are relative to one another, or how far apart they are from one another. Further still, the robot pose view 172 can also illustrate where the actual robotic arms 42A and 42B might be positioned or located relative to the inside the cavity of the patient that are to the left and right of the robotic arms 42A and 42B, thereby providing the user with a spatial awareness of where the robotic arms 42A and 42B are within the cavity, and where they are relative to anatomical features within the cavity. As noted above, because the full length of the robotic arms 42A and 42B are not visible in the live video footage 168, the simulated robotic arms 165 and 166 can provide the user with the spatial awareness to know how close or far apart the actual robotic arms 42A and 42B are from one another. The view provided by the robot pose view 172 is a view as if the user were looking at a field of the inside of the cavity. The robot pose view 172 provides the user with the spatial awareness to know how close the virtual elbows 128 are relative to one another if the user manipulates the right hand controller 202 and the left hand controller in such a way that the virtual elbows 128 are brought closer together, as well as how close the actual robotic arms 42A and 42B are to one another. For example, as the user manipulates the left hand controller 201 and the right hand controller 202 to straighten the robotic arms 42A and 42B, simulated robotic arms 166 and 165 will become parallel to one another and the distance between the elbow of simulated robotic arm 165 and the elbow of the simulated robotic arm 166 decreases. Conversely as the user manipulates the left hand controller 201 and the right hand controller 202 to bend the robotic arms 42A and 42B, so that the distance between the virtual elbows 128 of the robotic arms 42A and 42B are is further apart, simulated robotic arms 166 and 165 will not be parallel to one another and the distance between the elbow of simulated robotic arm 165 and the elbow of the simulated robotic arm 166 will increase. The robot pose views 171 and 172 provide the user with the spatial awareness during a surgical procedure, because the live video footage 168 does not provide visualization of the entire length of the robotic arms 42A and 42B.

In FIG. 9, the simulated robotic arms 191 and 192 are shown as being within the camera assembly 44 field of view associated with frustum 151, which provides the user with a situational awareness and spatial awareness of where the robotic arm 42B and the robotic arm 42A are located or positioned within a portion of the actual cavity captured of the patient. The camera view associated with the robot pose view 171 is a simulated view of the robotic arm 42B and the robotic arm 42A as if the user were viewing the actual view of the robotic arm 42B and the robotic arm 42A from a side view within the cavity of the patient.

As noted above, the camera view can be the camera assembly 44 field of view which is the frustum 167. That is, the robot pose view 171 provides the user with a side view of simulated robotic arms 191 and 192, which are simulated views corresponding to the robotic arm 42B and the robotic arm 42A respectively.

In some embodiments, the graphical user interface 150 can display live video footage 168 from a single vantage point including a field of view of the cavity and the robotic arm 42B and the robotic arm 42A relative to different areas within the cavity as shown in FIG. 9. As a result, the user might not always be able to determine how the virtual elbows 128 of the robotic arm 42B and the robotic arm 42A are positioned. This is because the camera assembly 44 might not always include video footage of the virtual elbows 128 of the robotic arm 42B and video footage of the elbow of the robotic arm 42A, and therefore the user may not be able to determine how to adjust the right hand controller 202 and left hand controller 201 if they wish to maneuver within the cavity of the patient. The simulated view of the robotic arm 42B (robotic arm 191) and the simulated view of the robotic arm 42A (robotic arm 192) provides the user with a view point that allows the user to determine the positioning of the virtual elbows 128 of the robotic arm 42A and the robotic arm 42B because the left situational awareness camera view panel includes a simulated field of view of the entire length of the robotic arms 191 and 192. Because the simulated field of view of the robot pose view 171 includes a view of the virtual elbows 128 of robotic arms 191 and 192, the user can adjust the positioning of the robotic arm 42B and the robotic arm 42A by manipulating the left hand controller 201 and the right hand controller 202, and watching how the robotic arms 191 and 192 move in accordance with the manipulation of the left hand controller 201 and the right hand controller 202.

The graphical user interface 150 can include the robot pose view 172 within which there is a frustum 167 that is the field of view, of the camera assembly 44, associated with a portion of the cavity of the patient, and the robotic arms 165 and 166 with a simulated camera 158 and simulated robotic supporting arm supporting the robotic arms 165 and 166.

In FIG. 9 the simulated robotic arms 165 and 166 are shown as being within the frustum 167, which is representative of location and positioning of the robotic arm 42B and the robotic arm 42A within the actual cavity of the patient. The view shown in the robot pose view 172 is a simulated view of the robotic arm 42B and the robotic arm 42A as if the user were viewing the robotic arm 42B and the robotic arm 42A from a top down view within the cavity of the patient. That is, the robot pose view 172 provides the user with a top down view of the simulated robotic arms 165 and 166, which are simulated views corresponding to the robotic arm 42B and the robotic arm 42A, respectively. The top down view provides the user with the ability to maintain a certain level of situational awareness of the robotic arm 42B and the robotic arm 42A as the user is performing a procedure within the cavity. The view of the simulated robotic arm 165 corresponding to the robotic arm 42B and the view of simulated robotic arm 166 corresponding to the robotic arm 42A provides the user with a top view perspective that allows them to determine the positioning of the robotic arm 42B and the robotic arm 42A, because the robot pose view 172 includes a simulated top-down field of view of the robotic arms 165 and 166, the camera 158, as well as support arm of the robotic assembly. Because the simulated field of view of the camera assembly 44 as outlined by the frustum 167 includes a top-down view of the simulated robotic arms 165 and 166, the user can adjust the positioning of the robotic arm 42B and the robotic arm 42A by manipulating the left hand controller 201 and the right hand controller 202, and watching how the simulated robotic arms 165 and 166 move forward or move backward within a portion of the cavity within the frustum 167 in accordance with the manipulation of the left hand controller 201 and the right hand controller 202.

The simulated view of the robotic arms 42B and 42A in the robot pose views 171 and 172 is automatically updated to stay centered on the trocar 50 while maintaining the robotic arms 42B and 42A in view. In some embodiments, this can be accomplished based on one or more sensors, from the sensing and tracking module 16 that are on the robotic arms 42B and 42A, providing information to the right hand controller 202 and the left hand controller 201. The sensors can be an encoder or hall effect sensor or other suitable sensor.

FIG. 9 depicts the graphical user interface 150 with the surgical robotic system 10 in an arm engagement mode. The arm engagement mode is an initialization process that guides the user to place the hand controllers 17 into the proper state to match the current state of the robotic arm assembly 42 to prevent unexpected motion from occurring prior to the robotic arm assembly 42 tracking the hand controllers 17. The details of a user involved arm engagement process are discussed in more detail below. The robot pose views 171 and 172 can provide the user with some situational awareness and spatial awareness about the orientation of the robotic arms 42A and 42B.

In some embodiments, the arm engagement process includes the process of engaging the right hand of the user with the right hand controller 202 and engaging the left hand of the user with the left hand controller 201 of the surgical robotic system 10 to ensure that the user places the right hand controller 202 and the left hand controller 201 into a proper state to match the current state of the robotic arm 42A and the robotic arm 42B in such a way that no unexpected motion occurs when the sensing and tracking module 16 begins tracking the right hand controller 202 and the left hand controller 201. This can be accomplished by guiding the user to place their right arm and hand into the correct position and orientation with respect to the right hand controller 202 and guiding the user to place their left arm and hand into the correct position and orientation with respect to the left hand controller 201. The user's right arm and left arm can be referred to as a “matching human right arm” and a “matching human left arm” respectively, and the user's right hand and left hand can be referred to as a “matching human right hand” and a “matching human left hand” respectively.

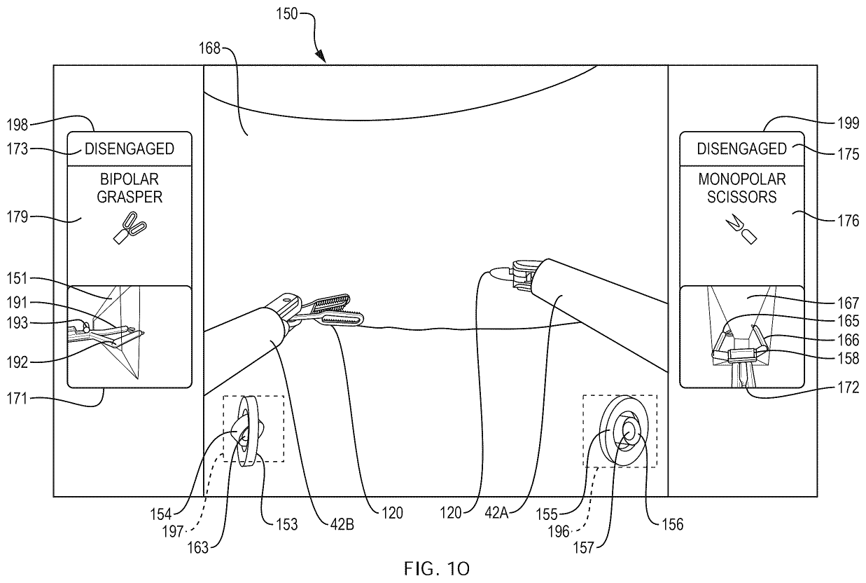

The process of engaging the user's right hand with the right hand controller 202 also ensures that an instrument 162 (instrument tip, or end effector), for example a grasper coupled to the robotic arm 42B as shown in FIG. 10, does not drop a surgical item, such as a suture or tissue, once the user is engaged with the robotic assembly, and begins to control the robotic arm assembly 42. Upon pressing or otherwise manipulating an engagement button or similar input on a hand controller (the right hand controller 202 or the left hand controller 201), the robotic surgical system enters the “intent to engage” mode. This in turn generates a signal to display, on the graphical user interface 150, an engagement guidance cue 197 and an engagement guidance cue 196 such as a matching human engagement ring 154 and an engagement ring 153 and a matching human engagement ring 156 and an engagement ring 155.

In some embodiments, there can be three engagement guidance cues, including a position cue, orientation cue and a grasper aperture cue. However, in some embodiments the position cue can be disabled when the robotic arms assembly is operating in autotrack mode. The autotrack mode enables the user to properly engage with the hand controllers without having to position the matching human engagement ring 154 inside the engagement ring 153 and the matching human engagement ring 156 inside the engagement ring 155. The matching human engagement ring 154 indicates the position of a matching human left arm and matching human left hand of the user relative to the position of the robotic arm 42B. The position of the matching human left arm and matching human left hand can be determined based on the positioning of the matching human left hand relative to the left hand controller 201. The matching human engagement ring 155 indicates the position of a matching human right arm and matching human right hand of the user relative to the position of the robotic arm 42A. The position of the matching human right arm and matching human right hand can be determined based on the positioning of the matching human right hand relative to the right hand controller 202. When the autotrack mode is enabled the user is able to engage with the hand controllers from any position, because the sensing and tracking module 16 tracks the position of the hand controllers that user's matching human left arm and matching human left hand and the user's matching human right arm and matching right hand are in contact with, as opposed to the user having to position their matching human arms and hands in the correct position relative to the hand controllers in order to engage with the robotic arm assembly 42.

Orientation cues can be a part of the engagement rings 153, 154, 155, and 156 which make up the position cue. In some embodiments, the matching human engagement ring 154, the engagement ring 153, the matching human engagement ring 156, and the engagement ring 155 are colored with four similar shades, each for a corresponding quadrant. The orientation cues correspond to the four similar shades. For example, a first orientation cue of the engagement rings can be displayed with a first color (e.g., white), a second orientation cue of the engagement rings can be displayed with a second color (e.g., lavender), a third orientation cue of the engagement rings can be displayed with a third color (e.g., teal), and a fourth orientation cue of the engagement rings can be displayed with a fourth color (e.g., white). When the user aligns the orientation cues of the matching human engagement ring 154 and the engagement ring 153, the user's matching human left arm and matching human left hand are oriented in the same way as the robotic arm 42B. When the user aligns the orientation cue of the matching human engagement ring 155 and the engagement ring 156, the user's matching human right arm and matching human right hand are oriented in the same way as the robotic arm 42A.

In some embodiments, the grasper aperture cues 163 and 157 can be a sphere that is located relative to the matching human engagement rings 154 and 156 such that when the user has properly placed their matching human right arm and hand and their matching human left arm and hand in the correct position relative to a trigger on the right hand controller 202 and the left hand controller 201, the user is said to be engaged with the right hand controller 202 and the left hand controller 201. As a result the sphere moves into the center of the matching human engagement rings 154 and 156. For example, when the user has properly placed a trigger of the left hand controller 201 in the correct position using their matching human left hand in order to engage with the left hand controller 201, left sphere 163 moves into the center of the engagement ring 154. Similarly, when the user has properly placed a trigger of the right hand controller 202 in the correct position using their matching human right hand in order to engage with the right hand controller 202, the right sphere 157 moves into the center of the engagement ring 156.

Once the user manages to place their matching arms and hands close enough with respect to the orientation and grasper aperture cues 163 and 157 the matching human engagement rings 154 and 156 turn green to indicate to the user that the robotic arm assembly 42 automatically engages the users matching arms after a set delay. After the delay, and in response to the user pressing the engagement button on the right hand controller 202 or the left hand controller 201 the surgical robotic system can return the users matching human arm(s) to a ready state in which the guidance cues 197 and 196 are no longer displayed on the graphical user interface 150. This indicates to the user that they are in control of the robotic arm assembly 42.

The guidance cues 197 and 196 are dynamically responsive graphical user interface elements that move in response to movements produced by the user's matching human arm(s) and hand(s) as the user is attempting to engage with the robotic assembly. For instance, when the user manipulates the right hand controller 202 or the left hand controller 201, the sensing and tracking module 16 can determine the positioning and orientation of the right hand controller 202 or the left hand controller 201 in response to the movement of the user's matching human arm(s) and hands. The sensing and tracking module 16 generates the tracking and position data 34 which can be transmitted to the computing module 18 for processing by the processor 22 and presentation on the display 12 in the form of the dynamically responsive graphical user interface element guidance cues 197 and 196.

It should be noted that the engagement guides are described herein as rings, however, other geometric shapes and forms are suitable for display on the graphical user interface 150 to provide a visual representation of an engagement state to the user. The robot pose views 171 and 172 can provide the user with some situational and spatial awareness about the orientation of the robotic arms 42A and 42B.

In some embodiments, the process to place the hand controllers into the proper state to match the current state of the robotic arm assembly 42 to prevent unexpected motion from occurring prior to the robotic arm assembly 42 tracking the hand controllers can occur automatically without the need for a user in the loop as described above. In some embodiments, engagement may take place with a compressive motion of the paddles as explained above in the description of FIGS. 6A-8B, without the need for the GUI engagement elements described above. In particular, in this embodiment, an operator may put his/her head close to a display such that his/her head is within a certain distance of the display, and the operator can squeeze the paddles as disclosed herein (e.g., as illustrated in FIGS. 6A-8B) to engage an instrument control mode.

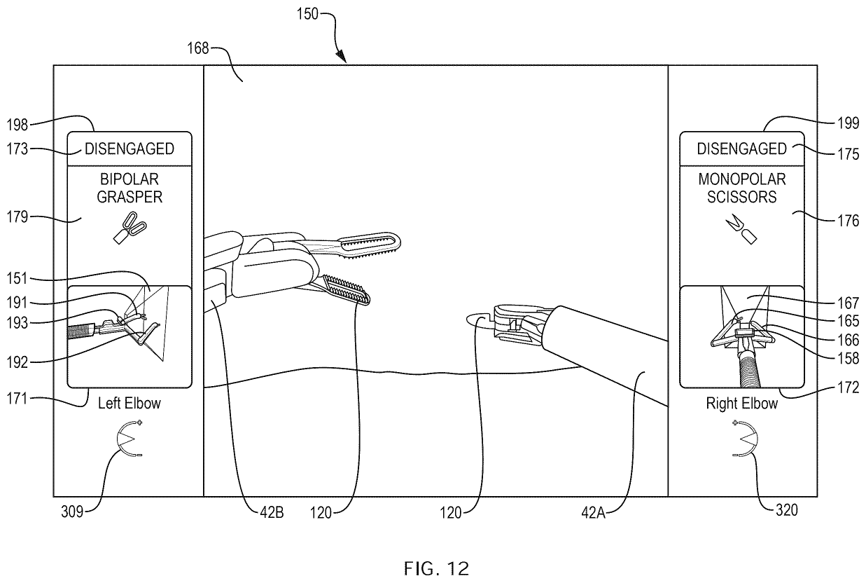

Turning to FIG. 11, the graphical user interface 150 can include an interactive menu 177 which can be a top level menu in which the user can select from one of three settings for elbow bias, brightness, and camera view. The user via the right hand controller 202 or the left hand controller 210 is able to interact with graphical user interface 150 to highlight or select one of the three settings, for example, and as shown in FIG. 11, “Elbow Bias” setting icon is highlighted. This particular setting can be selected by the user in response to the user manipulating the right hand controller 202 or the left hand controller 201 in order to access the bias associated with the robotic arm 42A or left robotic arm 162. In some embodiments, the user can manipulate touch input device 241 or touch input device 242 in order to access the Elbow Bias menu setting icon. After the user depresses the touch input device 241 or the touch input device 242, while the Elbow Bias menu setting icon is highlighted, the graphical user interface 150 as depicted in FIG. 12 displays a “Left Elbow” bias icon 309 in the left pillar box 198 associated with the bias of the virtual elbow 128 of the robotic arm 42B and a “Right Elbow” bias icon 320 in the right pillar box 199 associated with the bias of the virtual elbow 128 of the right robotic arm 166.

The “Left Elbow” bias icon 309 is a dynamically responsive graphical user interface element that updates in real time in response to the user adjusting the bias of the virtual elbow 128 associated with the robotic arm 42B. The “Left Elbow” bias icon 309 can be rendered to include a semicircle with a plus sign and a negative sign. The plus sign can indicate that the virtual elbow 128 associated with the robotic arm 42B, is above a neutral or default position. The negative sign can indicate that the virtual elbow 128 associated with the robotic arm 42B is below a neutral or default position. The Left Elbow bias icon 309 can change in response to the user manipulating one or more buttons on the left hand controller 201. Similarly, the graphical user interface 150 can include a “Right Elbow” bias icon 320. The “Right Elbow” bias icon 320 is a dynamically responsive graphical user interface element that updates in real time in response to the user adjusting the bias of the virtual elbow 128 associated with the robotic arm 42A. The “Right Elbow” bias icon 320 includes a semicircle with a plus sign and a negative sign. The plus sign can indicate that the virtual elbow 128, associated with the robotic arm 42A is above a neutral or default position. The negative sign can indicate that the virtual elbow 128 associated with the robotic arm 42A is below a neutral or default position. The Right Elbow bias icon 320 can change in response to the user manipulating one or more buttons on the right hand controller 202. As the user interacts with the left hand controller 201 or right hand controller 202 in order to bias the robotic arm 42B or the robotic arm 42A in a positive or negative direction, the robotic arms 191 and 192 as well as the robotic arms 165 and 166 also bias in accordance with the bias applied to the robotic arm 42B and the robotic arm 42A.

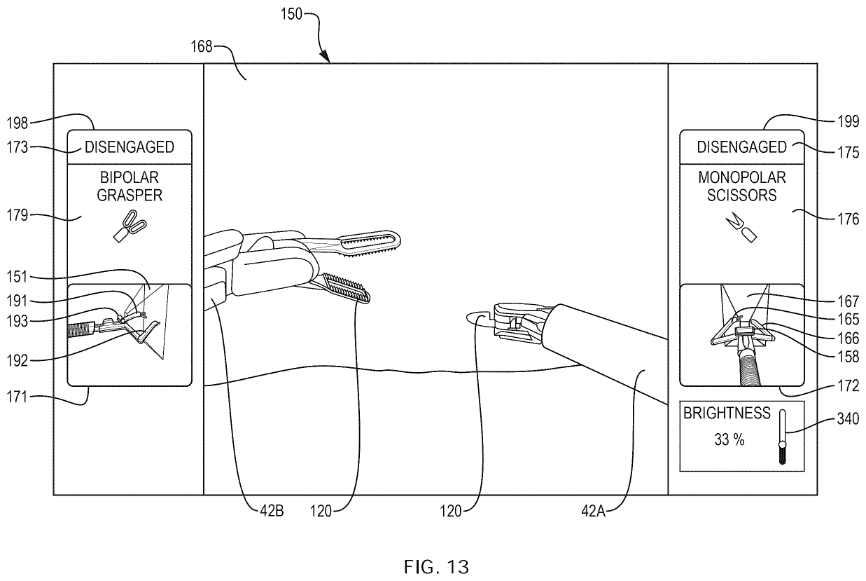

FIG. 13 depicts the graphical user interface 150 after the user manipulates one or more buttons or switches on the right hand controller 202 or left hand controller 201, the intensity of the brightness inside the cavity of patient can be changed. This can be reflected in the intensity of the brightness in the live video footage 168. Selection of the brightness menu 177 from the graphical user interface 150 allows the user to adjust the light sources (such as light emitting diodes (LEDs)) in or associated with the camera assembly 44. Selection of the brightness menu 177 causes the graphical user interface 150 to render a dynamically responsive graphical user interface element “Brightness” icon 340 that is responsive to input from the user to adjust the brightness of the light sources of the camera assembly 44. The dynamically responsive graphical user interface element brightness icon 340 includes a slider element 342 and a percent brightness scale 344. The user can adjust the brightness of the light sources of the camera assembly 44 by sliding the slider element 342 up or down. In turn, the percent brightness scale 344 updates based on a position of the slider element 342, which corresponds to the brightness of the light sources of the camera assembly 44. In some embodiments there can be an automatic brightness control that can employ both the LEDs and the gain (sensitivity) of an imaging sensor in the imaging devices or cameras disclosed herein.

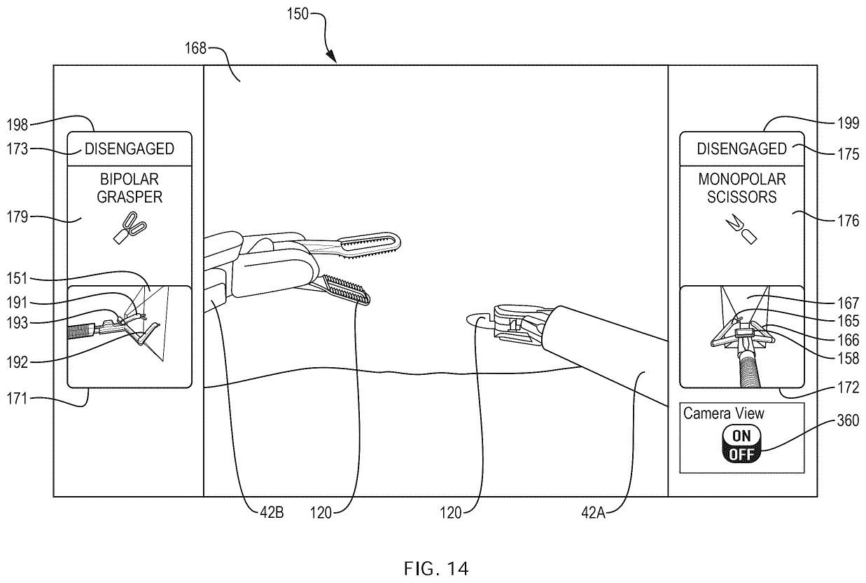

FIG. 14 depicts the graphical user interface 150 after the user selects the camera view menu 177. The camera view menu 177 allows the user to toggle the camera view from “On” to “Off” and vice versa using a switch of the “Camera View” icon 360. If the user uses the right hand controller 202 or left hand controller 201 to toggle the “Camera View” icon 360, then the graphical user 150 in response can turn the camera view in the robotic pose view 171 and the robotic pose view 172 “On” or “Off”. As noted above the camera view can be the camera's field of view which is the frustum 151 or frustum 167. That is, when the Camera View icon 360 is in the “On” position the frustum 151 and the frustum 167 will be displayed. When the Camera View icon 360 is in the “Off” position the frustum 151 and the frustum 167 will not be displayed.

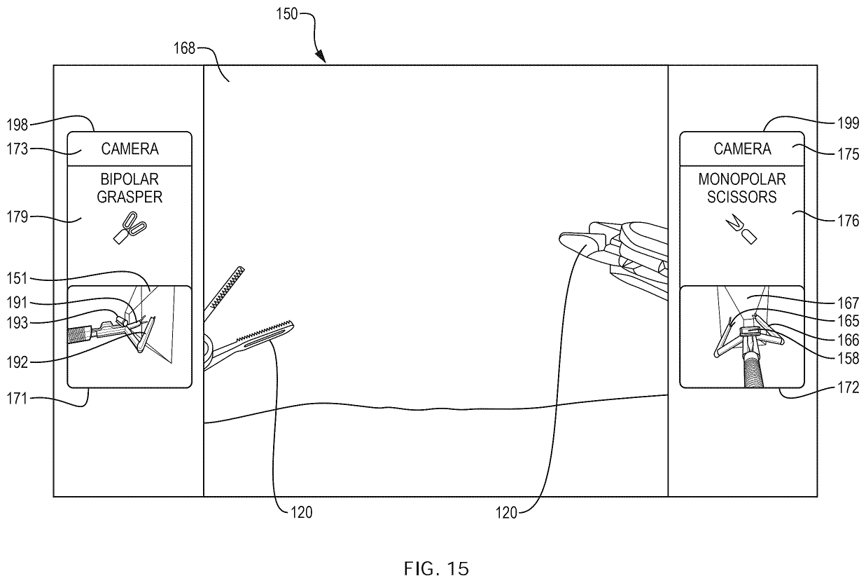

FIG. 15 depicts the graphical user interface 150 when the surgical robotic system 10 is in a camera mode 175 setting. In camera mode 175, movement (e.g., translation and/or rotation of one of the hand controllers 17 causes a corresponding movement (e.g., translation and/or rotation) of the camera assembly 44. A movement of the camera assembly 44 can include, but is not limited to a forward/backward translation, a vertical translation, a lateral translation, a yaw, a pitch, a roll, or any combination of the aforementioned. In the camera mode 175, instrument tips 120 of the robotic arms 42A and 42B remain stationary, but other portions of the robotic arms 42A and 42B may move to accomplish the corresponding motion of the camera assembly. For example, the virtual chest 140 of the robotic arms assembly 42 may need to translate and/or change its orientation to achieve the movement of the camera assembly 44. By keeping the instrument tips 120 in a fixed position and orientation, the view control mode enables an operator to frame a particular view, such as a view of a portion of the internal cavity in which a procedure is being performed, while not moving the instrument tip 120 or any tissue in contact therewith. The camera mode can have 3 degrees of freedom for controlling. In some embodiments, the direction of the hand controller movement can be opposite to the direction of chest movement. In some embodiments, paddles 1021-1024, 1021′-1024, and/or finger loops 1061-1064 of hand controllers 1001, 1002, 1001′, 1002′ can be automatically adjusted to be aligned with instrument tips and/or end effectors at all time.

For example, in some embodiments, when the surgical robotic system 10 is in the camera view mode, in response to a first movement including a first translation and a first rotation of the right hand controller 202 or the left hand controller 201, a position and an orientation of each instrument tip 120 is held while the camera assembly 44 and/or the chest are moved by a corresponding first movement including a corresponding first translation and a corresponding first rotation relative to the displayed camera assembly 44 view to maintain an angular deviation between the line from the center of the virtual chest to the average instrument tip position and the normal to the virtual chest within the acceptable angular deviation range.

In some embodiments, a camera view mode is entered and exited in response to an user input (e.g., an operator input via a foot pedal, which may be a dedicated camera control foot pedal). In some embodiments, when the camera view mode is exited, a framing or view is maintained, meaning that a position and an orientation of the camera assembly 44 is maintained.

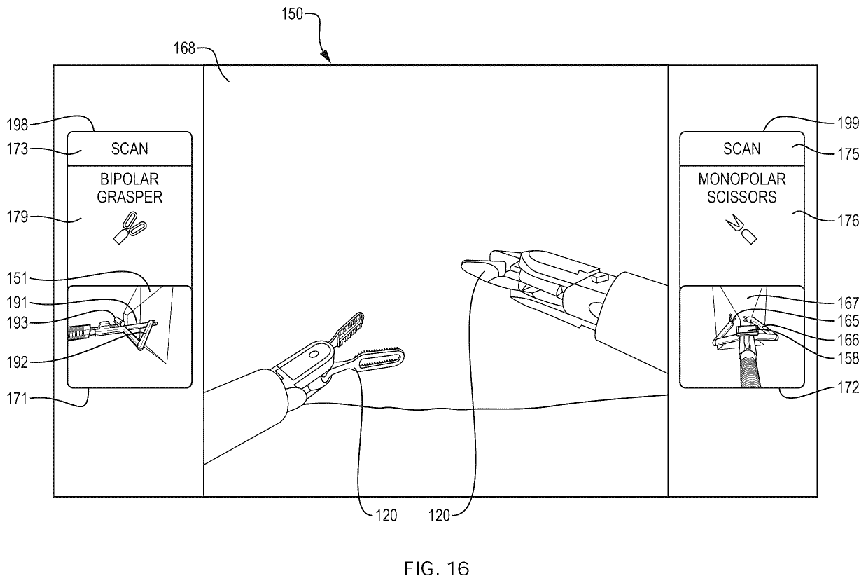

FIG. 16 depicts the graphical user interface 150 when the surgical robotic system 10 is in a scan mode 175 setting. In some embodiments, the surgical robotic system 10 can employ or provide a scan mode 175, which may also be described as a “scan control mode”, or a “scanning mode” herein. In the scan mode 175, rotation of one of the hand controllers 201 or 202 causes a corresponding rotation of the camera assembly 44 (e.g., a yaw rotation, a pitch rotation, a roll rotation, or a combination of the aforementioned) to change a view provided by the camera assembly 44. In the scan mode 175, movement of either of the hand controllers 201 or 202 causes no movement of the robotic arms 42A or 42B and causes no movement of the virtual chest 140 of the robotic arm assembly 42. The scan mode 175 may be used to quickly survey an interior body cavity, to check the virtual elbow 128 position of the robotic arms 42A or 42B, and or to look for surgical materials.

In some embodiments, the scan mode 175 is entered and excited, which may be described as the scan mode 175 being activated and deactivated, using an input from one or both of the hand controllers 201 or 202 using an input from one or both of the hand controllers 201 or 202 in combination with an input from the foot pedals 203 (e.g., via input touch device 241 in FIG. 6A). In some embodiments, when the scan mode 175 is exited, an orientation and position of the camera assembly 44 returns to the orientation and position that the camera assembly 44 had when the scan mode 175 was entered.

In some embodiments, when the surgical robotic system 10 is in the scan mode 175, in response to a first movement including a first rotation of the right hand controller 202 or the left hand controller 201, the robotic arm assembly 42 is held stationary while the camera assembly 44 is rotated in a corresponding first rotation relative to a view of the camera assembly 44 displayed on the display 12, which is a displayed camera view. In some embodiments, a scan mode can have 2 degrees of freedom for controlling without rolling degree of freedom.

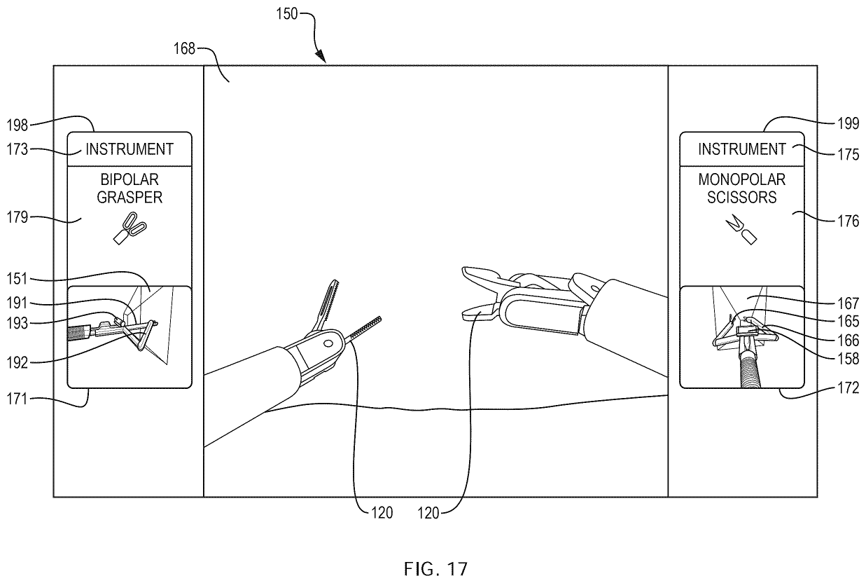

FIG. 17 depicts the graphical user interface 150 when the surgical robotic arms assembly 10 is in an instrument mode 175 settings. In some embodiments the surgical robotic arms assembly 10 can employ or provide an instrument control mode 175, which may be described as an “instrument mode” herein. In the instrument mode 175, the surgical robotic system 10 identifies movement (e.g., translation and/or rotation) of each hand controller 201 or 202 and moves (e.g., translates and/or rotates) an instrument tip 120 on a distal end of the corresponding robotic arm 42A or 42B in a corresponding matter. In the instrument control 175 mode, the surgical robotic system 10 may cause an instrument tip 120 to move in a manner directly proportional to movement of a corresponding hand controller 201 or 202.

This may be described as motion including translation and/or rotation of the instrument tip 120 of a robotic arm 42A or 42B being directly controlled by motion of respective hand controller 201 or 202. For example, translating a hand controller 201 or 202 in a direction by an amount causes the corresponding instrument tip 120 for the corresponding robotic arm 42A or 42B to move in a corresponding direction (i.e., in the same direction with respect to a view from the camera assembly 44 displayed to the operator) by a corresponding scaled down amount (e.g., where the scaling is based on the scale of the view from the camera assembly 44 displayed to the operator). As another example, rotating a hand controller 201 or 202 about an axis by an angle causes the corresponding instrument tip 120 for the corresponding robotic arm 42A 42B to rotate by a same angle or by a scaled angle about a corresponding axis (e.g., where the corresponding axis is a same axis with respect to the orientation of the view from the camera assembly 44 displayed to the operator). In the instrument mode 175, operator controls can be used to actuate instruments (e.g., via grasper controls of a hand controller, via foot pedal controls) as well as we as to move or change an orientation of instrument tips 120.

In the instrument mode 175, movement of the hand controllers 201 or 202 does not change a position and does not change an orientation of the camera assembly 44 (e.g., the camera assembly 44 orientation and position may remain fixed) and does not change a position or an orientation of the virtual chest 140. In other words, the instrument mode 175 does not reposition or reorient the camera assembly 44 or the virtual chest 140. The instrument control mode 175 is useful for manipulating the instrument tips 120 within a working area of an internal body cavity that is accessible without moving a virtual chest 140 of the robotic arm assembly 42.

In some embodiments, the operator can enable or disable the instrument control mode 175 via either or both of the hand controllers 201 or 202. In some embodiments, an instrument mode 175 is engaged and disengaged using an input control from a hand controller 201 or 202 (e.g., by pressing a button, such as button 233 in FIG. 6A, or interacting with a touch input device). When the instrument control mode 175 is disengaged, any movement of a hand controller 201 or 202 does not cause any corresponding movement of the associated instrument tip 120. In some embodiments, when the surgical robotic system 10 is in a disengaged state, an information portion of the graphical user interface 150 can indicate that the current state is disengaged. In some embodiments, engaging the clutch causes an information panel of the graphical user interface 150 to identify that the clutch is engaged (e.g., via text, color, or any other graphical indicator).

In some embodiments an operator may put his/her head close to a display such that his/her head is within a certain distance of a display, and the operator can squeeze the paddles as disclosed herein (e.g., as illustrated in FIGS. 6A-8B) to engage an instrument control mode. The operator may pull his/her head away from the display and therefor away from the sensor, that determines how close his/her head is to the display, in order to disengage the instrument control mode.

In some embodiments, the instrument control mode 175 is a default control mode that the surgical robotic system 10 enters when another control mode is exited.

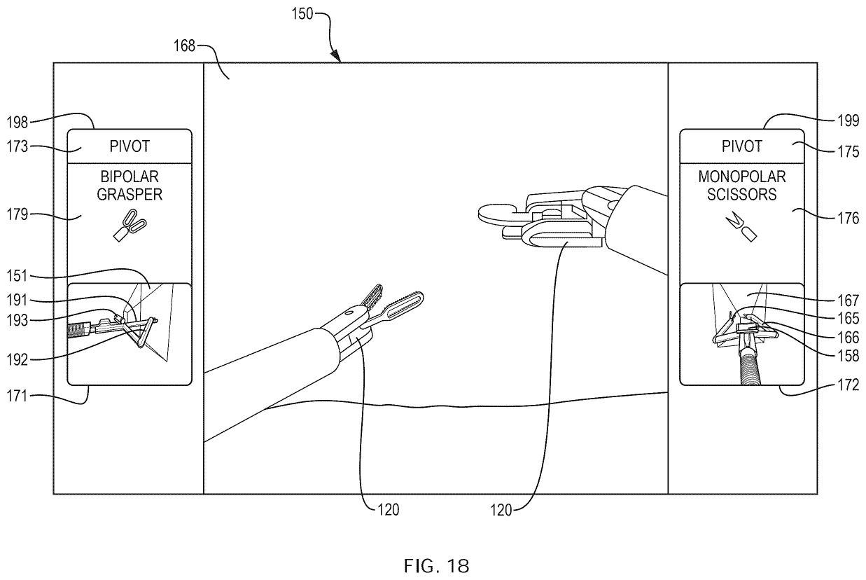

FIG. 18 depicts the graphical user interface 150 when the robotic arm assembly 42 is in a pivot mode 175 setting. In some embodiments, the surgical robotic system 10 can implement a pivot control mode, which may also be referred to as a “pivot mode” 175 herein. The pivot mode 175 enables a user to control the positions and orientations of the instrument tips 120 by corresponding movements of the hand controllers 201 or 202, while changing an orientation of the camera assembly 44 or an orientation of the camera assembly 44 and an orientation of the virtual chest 140 to maintain a center of view of the camera assembly 44 on the average instrument tip 120 position. For example, when in the pivot mode 175, in response to a first movement including a first translation and a first rotation of the right hand controller 202 or the left hand controller 201, a corresponding instrument tip 120 is moved by a corresponding first movement including a corresponding first translation and a corresponding first rotation relative to the displayed camera view (frustum 151 or 167), the camera assembly 44 is rotated to center the view of the camera assembly 44 on the average instrument tip 120 position causing a change in the orientation of the camera assembly 44 or a change in the orientation of the camera assembly 44 and a change in the orientation of the virtual chest 140 to maintain the view of the camera assembly 44 centered on the average instrument tip 120 position and to maintain an angular deviation between the line from the center of the virtual chest 140 to the average instrument tip 120 position and the normal to the virtual chest 140 within the acceptable angular deviation range.

In some embodiments, engagement of a foot pedals 203 activates the pivot mode 175. In some embodiments, engagement of an input function on a hand controller 201 or 202 activates the pivot mode 175. In some embodiments, a pivot mode may be deprecated in the surgical robotic system. For example, functionalities of a pivot mode can be consolidated into a camera mode.

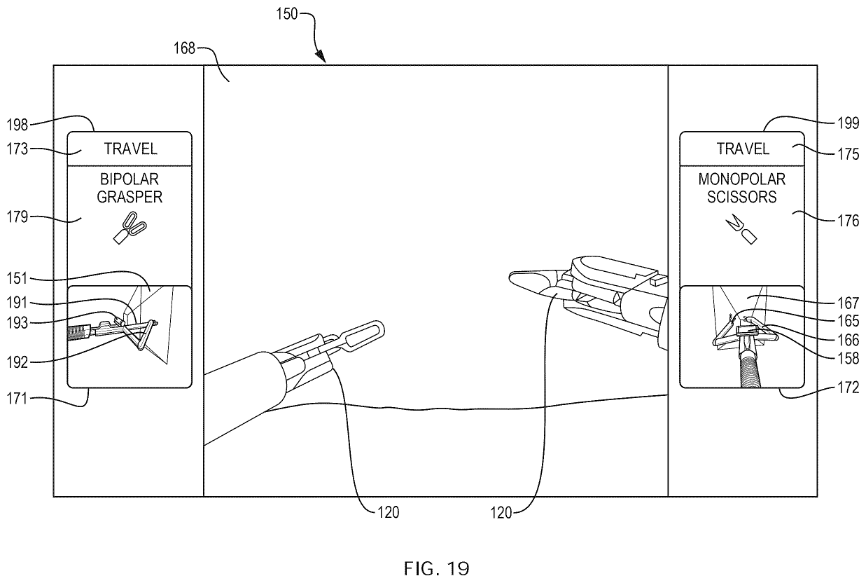

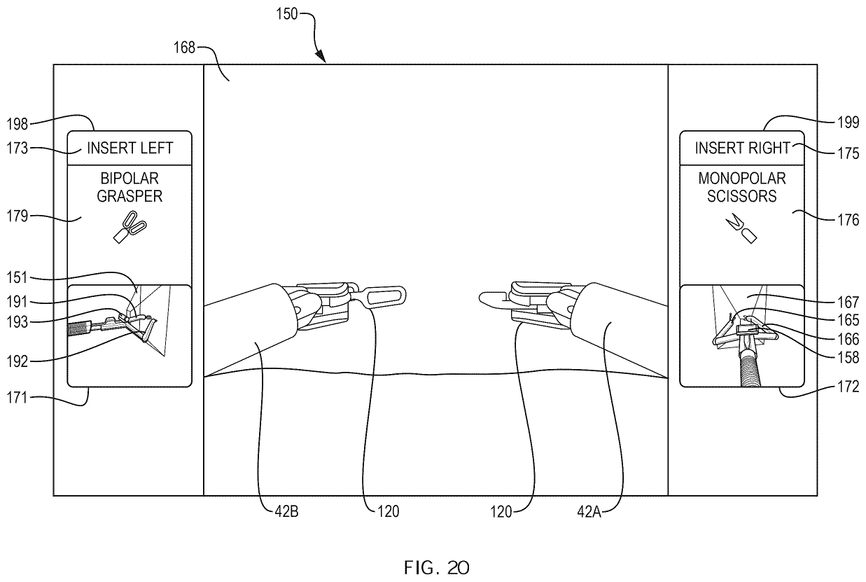

FIG. 19 depicts the graphical user interface 150 when the robotic arm assembly 42 is in a travel control mode 175 setting. When the travel control mode 175 is activated, movements of the left hand controller 201 and the right hand controller 202 are translated into corresponding movements of end effectors or instrument tips 120 of the robotic arm assembly 42. Similar to the instrument control mode 175, instrument and tools can be actuated in the travel control mode 175. Unlike the instrument control mode 175, in the travel control mode 175, the camera assembly 44 and the virtual chest 140 track a midpoint between an instrument tip 120 or tips of the robotic arm 42A and an instrument tip 120 or tips of the robotic arm 42B. Unlike the instrument control mode 175, in the travel mode 175, movement of the hand controllers 201 or 202 can also cause displacement and/or a change in orientation of the virtual chest 140 of the robotic assembly 44, enabling the robotic arms 42A and 42B assembly to “travel”. This may be described as the instrument tips 120 directing or leading movement through an interior body cavity. For example in some embodiments the surgical robotic system 10 can establish a cone of movement (e.g., an acceptable range for a distance of the instrument tips from the virtual chest 140 of the robotic arm assembly 42 position, and an acceptable range of deviation for a line connecting the center of the virtual chest 140 to the instrument tips 120 from a normal of the virtual chest 140), and when the instrument tips 120 or end effectors would exceed the cone of movement, the virtual chest 140 and robotic arms 42A and 42B of the robotic system 10 automatically move to keep the instrument tips 120 within the cone of movement (see FIG. 20). The cone of movement may not have a constant length or a constant angular range, but the length and the angular range may vary during use based on some other parameters of the surgical robotic system 10, or based on currently selected features and options of the surgical robotic system 10 (e.g., based on a zoom of the camera view displayed).

For example, in some embodiments, when in the travel mode 175, in response to a first movement including a first translation and a first rotation of the right hand controller 202 or the left hand controller 201, a corresponding instrument tip 120 is moved by a corresponding first movement including a corresponding first translation and a corresponding first rotation relative to the displayed camera view (frustum 151 or 167) while the camera assembly 44 is rotated to center the view of the camera assembly 44 on a position of a midpoint between instrument tips 120 of the robotic arms 42A or 42B of the robotic arm assembly 42, which is an average tip position, while robotic arm assembly 42 is translated to a chest point of the virtual chest 140, the robotic arm assembly 42 is rotated to rotate the virtual chest 140, or both, to maintain a distance between the center of the virtual chest 140 and the average tip position 120 in an acceptable distance range, and to maintain an angular deviation between a line from the virtual chest 140 point to the average tip position and a normal to the virtual chest 140 within an acceptable angular deviation range

In some embodiments, the travel control mode 175 may be used to navigate the robotic arm assembly 42 to another location in an internal body cavity of a patient or to maintain visualization while a surgical task is performed. As noted above, in travel mode 175, the camera assembly 44 automatically tracks the midpoint between the instrument tips 120 during movement of the instrument tips 120. Accordingly, travel mode 175 may be useful for navigation and visualization for a task because the user will be able to maintain a consistent view of the instrument tip 120. This visualization may be valuable, for example, in procedures such as suturing the circumference of a mesh or creating a flap.

FIG. 20 depicts the graphical user interface 150 when the surgical robotic system 10 is an insertion mode setting. The graphical user interface 150 can display a camera view (frustum 151 or 167) of the robotic arms 42A and 42B, and the robotic arms 165 and 166, corresponding to the robotic arms 42B and 42A as they are being inserted into the patient. When the surgical robotic system 10 is in the insertion mode setting, the graphical user interface 150 can provide instructions to the user to enter the robotic arms 42A and 42B into the cavity of the patient in a straight (no bending of the robotic arms 42A and 42B) configuration until a predetermined insertion depth is met. Once the robotic arms 42A and 42B have achieved the predetermined depth, the robotic arms 42A and 42B either manually or automatically enter into a “procedure or surgical ready state”.

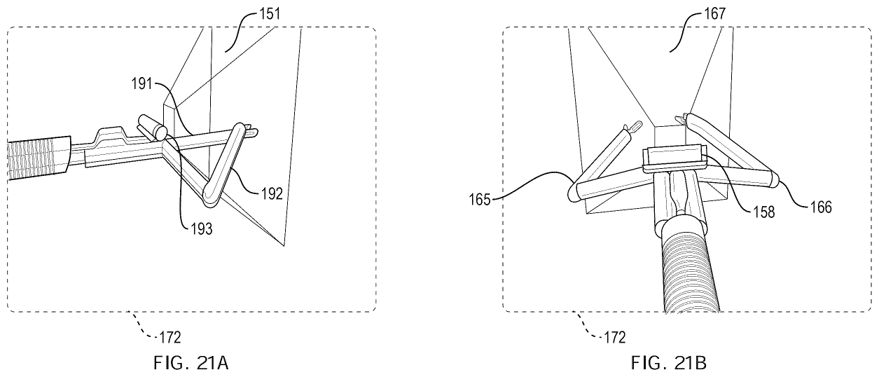

FIG. 21A is exploded close up view of the robot pose view 171 shown in the left pillar boxes 198 in FIGS. 9-20. And FIG. 21B is a close up view of the camera pose view 172 shown in the right pillar boxes 199 in FIGS. 9-20.

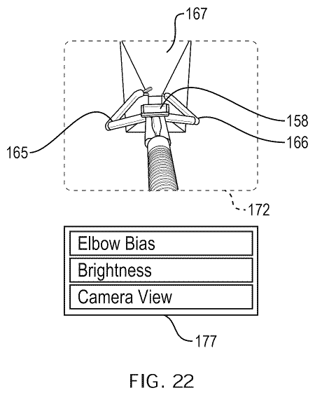

FIG. 22 is close up view of the camera pose view 172 and interactive menu 177 in FIG. 11.

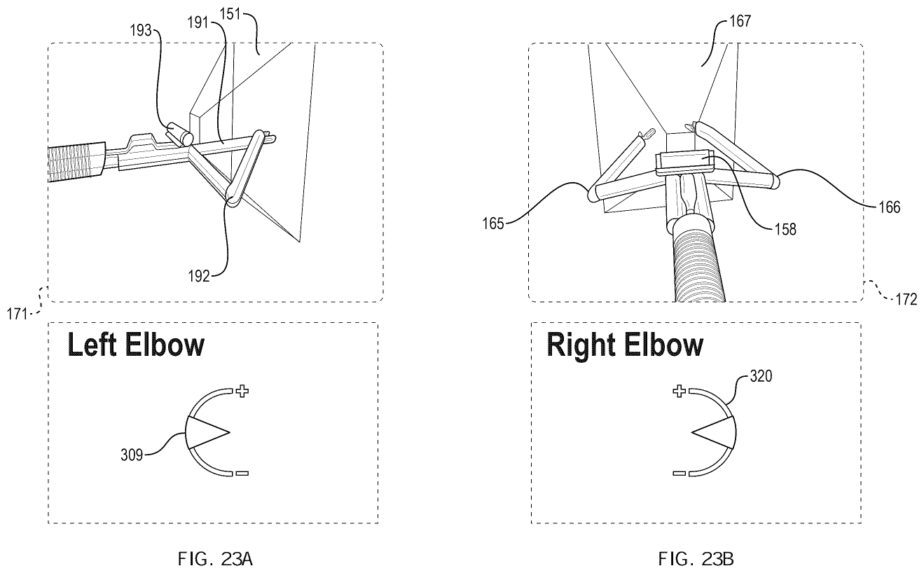

FIG. 23A is exploded close up view of the robot pose view 171 shown in the left pillar box 198 in FIG. 12. FIG. 23B is exploded close up view of the camera pose view 172 shown in the right pillar box 199 in FIG. 12.

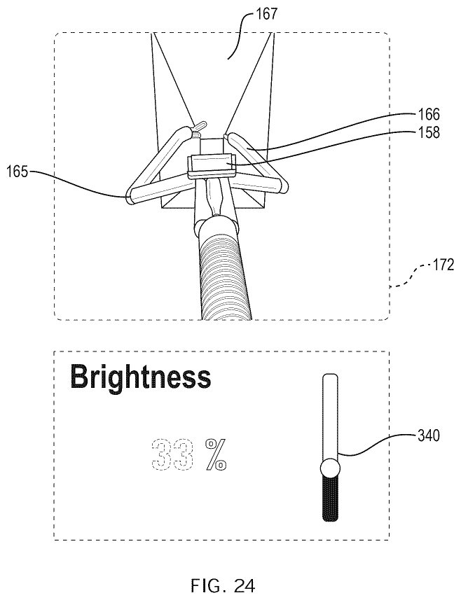

FIG. 24 is close up view of the robot pose view 172 show in the right pillar box 199 including an interactive menu for adjusting a camera brightness setting 340 of the surgical robotic system shown in FIG. 13.

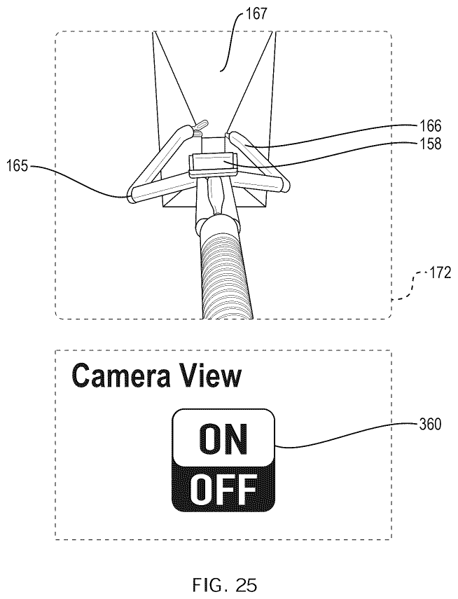

FIG. 25 is close up view of the robot pose view 172 shown in the right pillar box 199 including an interactive menu for toggling a camera view 360 in accordance with some embodiments.

FIG. 26 schematically depicts the graphical user interface 1100 including a camera view portion 150, which can be live video footage 168, displaying a view from the camera assembly and a menu 1120. A hand controller may be used by the operator to select an item listed in the menu 1120, such as by controlling a touch input device or other button or switch on the hand controllers. The operator can access the menu 1120 by pressing the second button 235 and navigating through a list of items in the menu 1120 using the first touch input device 242. In other embodiments the operator can access the menu 1120 by pressing the second button 1035′ and navigate through the list of items in the menu 1120 using the touch input device 1042′. The touch input device 1042′ may trigger a signal used to traverse the menu 1120 or highlight a portion of the menu 1120 when the menu 1120 is displayed or a menu mode is active by pressing the touch input device 1035′. When the second button 235 is selected the camera view portion 150 is shifted to the outermost left edge, or outermost right edge, of the display and the space occupied by the pillar boxes 198 and 199 are replaced by the menu 1120. For instance if the camera view portion 150 is shifted to the outermost left edge of the display, the menu 1120 will occupy the space once occupied by the pillar box 199. Alternatively if the camera view portion 150 is shifted to the outermost right edge of the display, the menu 1120 will occupy the space once occupied by the pillar box 198.

As an example, the operator can use the first touch input device 242 in order to select the “Adjust Elbow Bias” by moving the first touch input device 242 upward (forward), or downward (backward), until the “Adjust Elbow Bias” item is highlighted. When the first touch input device 242 is depressed while the “Adjust Elbow Bias” item is highlighted, the user interface 150 can change to the one shown in FIG. 12, where the operator can adjust the bias of the virtual elbow 128. As another example, the operator can use the first touch input device 242 in order to select the “Camera LED Brightness” by moving the first touch input device 242 upward (forward), or downward (backward), until the “Camera LED Brightness” item is highlighted. When the first touch input device 242 is depressed while the “Camera LED Brightness” item is highlighted, the user interface 150 can change to the one shown in FIG. 13, where the operator can adjust the brightness, or intensity, of the light inside the cavity of the patient.

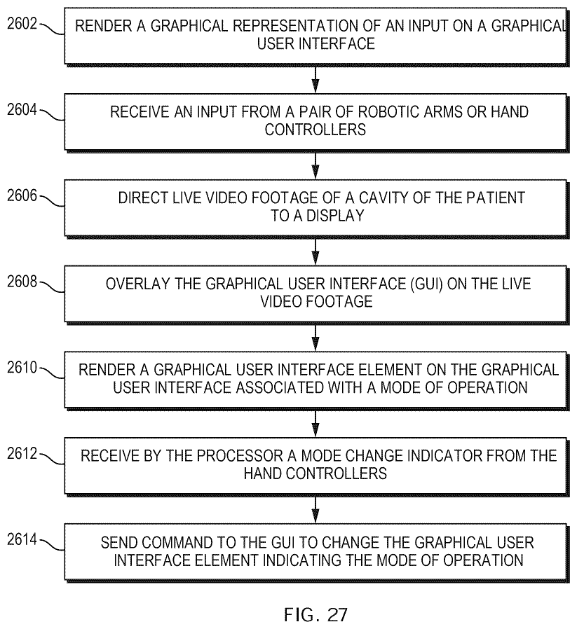

FIG. 27 is an example flowchart corresponding to changing a mode of operation of the surgical robotic system 10. The method can begin at block 2602, at which point the processor 22, can render a graphical representation of the input on the graphical user interface 150 in order to provide the use with the ability to visualize an engagement state between the hands of the user and the hand controllers or a spatial awareness of the robotic arms inside the patient. The graphical user interface could be graphical user interface 150, and can be rendered on display 12. This provides the user with the ability to determine how to engage their hands and arms with the hand controllers. The user can engage with the left hand controller 201 and the right hand controller 202 as explained above in reference to FIG. 10.

Additionally, or alternatively, the graphical user interface 150 can also show the robot pose views 171 and 172 to provide the user with some spatial awareness about the position and location of the right robotic arm 42A and the second robotic arm 42B relative to any area within the field of view of a camera of the camera assembly 44, which is depicted by frustums 151 and 172. Also as explained above, the robot pose views 171 and 172 can provide the user with some situational awareness and spatial awareness about the orientation of the robotic arms 42A and 42B.

At block 2604 the processor 22 of the computing module 18, can receive an input from the left hand controller 201 or the right hand controller 202 associated with the surgical robotic system 10. In some embodiments, the image renderer can perform the operation at block 2604. The input can be associated with an engagement state between a matching arm and hand of the user and one of the left hand controller 201 or the right hand controller 202 or a spatial awareness of the robotic arms inside a patient. For instance, in some embodiments, sensors associated with the sensing and tracking module 16 in one or both of the robotic arm 42A and the robotic arm 42B can be used to track the movement of the left hand controller 201 and the right hand controller 202.

In some embodiments, sensors in one or both of the first robotic arm and second robotic arm can be used by the system to determine a location in three-dimensional space of at least a portion of one robotic arm relative to a location in three-dimensional space of at least a portion of the other robotic arm. The sensors in the first robotic arm 42A and the second robotic arm 42B can generate data associated with the location in three-dimensional space of the first robotic arm 42A and second robotic arm 42B that can be input by the processor 22 and used by the processor 22 to generate a robot pose view similar to the robot pose views 171 and 172 and a corresponding camera view of the robot pose views 171 and 172. The pose views 171 and 172 provide a situational and spatial awareness for the user as discussed above with respect to FIG. 9.

At block 2606 the processor 22 can transmit live video footage 168 captured by the camera of the camera assembly 44 of a cavity of the patient to display 12. The processor 22 can then overlay the graphical user interface 150 on the live video footage 168 in block 2608.

At block 2610, the processor 22 can render a graphical user interface element on the graphical user interface 150 associated with a mode of operation. For instance, the processor 22 can render a camera mode 175 of operation of the surgical robotic system 10 as shown in FIG. 15. Should the processor 22 receive a change mode indicator from the right hand controller 202 or the left hand controller 201, the processor 22 can instruct the graphical user interface 150 to change the graphical user interface element from the camera mode 175 of operation to another mode of operation. For instance, the processor 22 can receive another change mode indicator, at block 2612, from the right hand controller 202 or the left hand controller 201, and the processor 22 can send a command to the graphical user interface 150 to render a graphical user interface element associated with the scan mode of operation as shown in FIG. 16. The processor 22 can also generate a signal that causes the surgical robotic system to exit the camera mode 175 and activate the scan mode of operation.

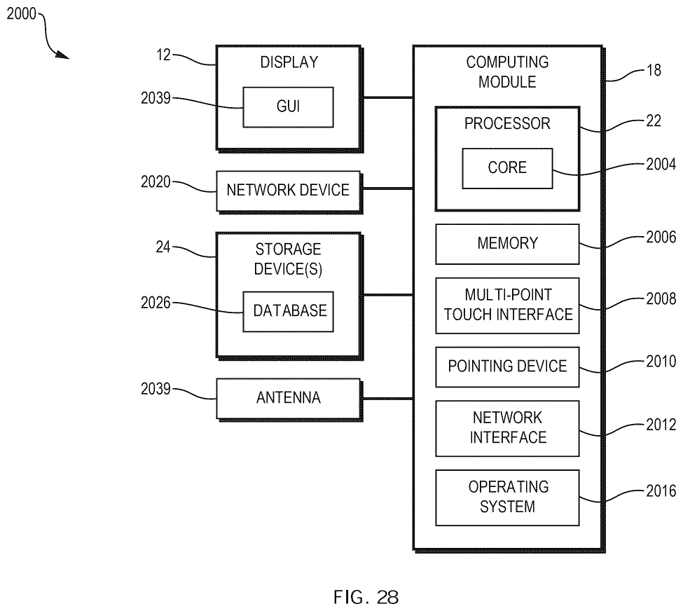

FIG. 28 schematically depicts an example computational environment 2000 that the surgical robotic system can be connected to in accordance with some embodiments.

Computing module 18 can be used to perform one or more steps of the methods provided by example embodiments. The computing module 18 includes one or more non-transitory computer-readable media for storing one or more computer-executable instructions or software for implementing example embodiments. The non-transitory computer-readable media can include, but are not limited to, one or more types of hardware memory, non-transitory tangible media (for example, one or more magnetic storage disks, one or more optical disks, one or more USB flashdrives), and the like. For example, memory 2006 included in the computing module 18 can store computer-readable and computer-executable instructions or software for implementing example embodiments. The computing module 18 also includes the processor 22 and associated core 2004, for executing computer-readable and computer-executable instructions or software stored in the memory 2006 and other programs for controlling system hardware. The processor 22 can be a single core processor or multiple core (2004) processor.

Memory 2006 can include a computer system memory or random access memory, such as DRAM, SRAM, EDO RAM, and the like. The memory 2006 can include other types of memory as well, or combinations thereof. A user can interact with the computing module 18 through the display 12, such as a touch screen display or computer monitor, which can display the graphical user interface (GUI) 39. The display 12 can also display other aspects, transducers and/or information or data associated with example embodiments. The computing module 18 can include other I/O devices for receiving input from a user, for example, a keyboard or any suitable multi-point touch interface 2008, a pointing device 2010 (e.g., a pen, stylus, mouse, or trackpad). The keyboard 2008 and the pointing device 2010 can be coupled to the visual display device 12. The computing module 18 can include other suitable conventional I/O peripherals.

The computing module 18 can also include one or more storage devices 24, such as a hard-drive, CD-ROM, or other computer readable media, for storing data and computer-readable instructions, applications, and/or software that implements example operations/steps of the surgical robotic system 10 as described herein, or portions thereof, which can be executed to generate GUI 39 on display 12. Example storage devices 24 can also store one or more databases for storing any suitable information required to implement example embodiments. The databases can be updated by a user or automatically at any suitable time to add, delete or update one or more items in the databases. Example storage device 24 can store one or more databases 2026 for storing provisioned data, and other data/information used to implement example embodiments of the systems and methods described herein.

The computing module 18 can include a network interface 2012 configured to interface via one or more network devices 2020 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 2012 can include a built-in network adapter, network interface card, PCMCIA network card, card bus network adapter, wireless network adapter, USB network adapter, modem or any other device suitable for interfacing the computing module 18 to any type of network capable of communication and performing the operations described herein. Moreover, the computing module 18 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing module 18 can run any operating system 2016, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In some embodiments, the operating system 2016 can be run in native mode or emulated mode. In some embodiments, the operating system 2016 can be run on one or more cloud machine instances.

The computing module 18 can also include an antenna 2030, where the antenna 2030 can transmit wireless transmissions a radio frequency (RF) front end and receive wireless transmissions from the RF front end.

What is claimed is:

1. A surgical robotic system comprising:
a camera assembly;
a display;
a robotic arm assembly having robotic arms;
hand controllers graspable by a user of the surgical robotic system to control the robotic arms and the camera assembly;
a memory storing one or more instructions;
a processor configured to or programmed to read the one or more instructions stored in the memory, the processor operationally coupled to the robotic arm assembly, the hand controllers and the camera assembly to:
receive input from the hand controllers,
render a graphical representation of the input on a graphical user interface (GUI) to provide the user with a visual representation of at least an engagement state between hands of the user and the hand controllers or a spatial awareness of the robotic arms within the cavity,
overlay the GUI on live video footage on the display,
render on the GUI, a graphical user interface element indicating a first mode of the surgical robotic system,
receive a mode change indicator from the hand controllers, and
in response to the mode change indicator:
instructing the GUI to change the graphical user interface element indicating the first mode, and
causing the surgical robotic system to exit the first mode, and activate a second mode.

2. The surgical robotic system of claim 1, wherein the display is responsive to the processor to render the GUI having one or more selectable menu items associated with control of the robotic arms or one or more instruments coupled to the robotic arms.

3. The surgical robotic system of claim 1, wherein the hand controllers include a plurality of buttons or touch inputs operable by the user of the surgical robotic system.

4. The surgical robotic system of claim 3, wherein the robotic arm assembly is communicatively coupled to the hand controllers and is responsive to selection of one or more of the plurality of buttons or touch inputs to control the robotic arms or one or more instruments coupled to the robotic arms.

5. The surgical robotic system of claim 2, wherein the display renders on the GUI one or more frustums associated with a view of the camera.

6. The surgical robotic system of claim 5, wherein the visual representation of the spatial awareness of the robotic arms includes one of the one or more frustums.

7. The surgical robotic system of claim 2, wherein the display further renders at least one dynamically responsive graphical user interface element to provide visual feedback to the user regarding the engagement state.

8. The surgical robotic system of claim 7, wherein the at least one dynamically responsive graphical user interface element changes in real time in response to a relationship between a movement of one of the hand controllers to a position of one of the robotic arms.

9. The surgical robotic system of claim 8, wherein a first portion of the at least one dynamically responsive graphical user interface element corresponds to a position and orientation of a first hand controller of the hand controllers, and a second portion of the at least one dynamically responsive graphical user interface element corresponds to a position and orientation of one of the robotic arms.

10. The surgical robotic system of claim 9, wherein a third portion of the at least one dynamically responsive graphical user interface element corresponds to the engagement state of a tool held in one of the robotic arms.

11. The surgical robotic system of claim 2, wherein the display further renders a representation of a first robotic arm of the robotic arms, and renders a representation of a second robotic arm of the robotic arms.

12. The surgical robotic system of claim 9, wherein the display further renders a representation of a grasper tool associated with one of the robotic arms.

13. The surgical robotic system of claim 9, wherein the display further renders a representation of a scissor tool associated with one of the robotic arms.

14. The surgical robotic system of claim 1, wherein the visual representation of at least the engagement state between the hands of the user of the hand controllers includes a first set of two concentric rings and a second set of two concentric rings.

15. The surgical robotic system of claim 1, wherein the processor is further configured to or programmed to read the one or more instructions stored in the memory to:
transmit the live video footage of a cavity of a patient from the camera assembly to the display.

16. A method of controlling a surgical robotic system comprising:
generating with a processor configured to or programmed to read one or more instructions stored in memory a graphical user interface (GUI) on a display of the surgical robotic system, the processor operationally coupled to hand controllers graspable by a user of the surgical robotic system, a robotic arm assembly having robotic arms, and a camera assembly,
displaying on the display live video footage captured by the camera assembly,
overlaying the GUI on the live video footage on the display,
receiving by the processor input from one or more buttons or touch inputs on the hand controllers to control the robotic arm assembly,
rendering a graphical representation of the input on the GUI to provide the user with a visual representation of at least an engagement state between hands of the user and the hand controllers or a spatial awareness of the robotic arms within a cavity to control the surgical robotic system,
rendering on the GUI a graphical user interface element indicating a first mode of the surgical robotic system,
receiving by the processor a mode change indicator from the hand controllers, and
responsive to the mode change indicator:
instructing the GUI to change the graphical user interface element indicating the first mode, and
causing the surgical robotic system to exit the first mode, and activate a second mode.

17. The method of claim 16, further comprising, rendering on the GUI one or more selectable menu items associated with control of the robotic arms or one or more instruments coupled to the robotic arms.

18. The method of claim 16, wherein the hand controllers include a plurality of buttons or touch inputs operable by the user of the surgical robotic system.

19. The method of claim 18, wherein the robotic arm assembly is communicatively coupled to the hand controllers and are responsive to selection of one or more of the plurality of buttons or touch inputs to control the robotic arms or one or more instruments coupled to the robotic arms.

20. The method of claim 16, further comprising, rendering on the GUI one or more surgical robotic pose views to display a simulated view of the robotic arms or camera field of view of the camera assembly.

21. The method of claim 20, wherein a visual representation of the robotic pose views includes a view frustum.

22. The method of claim 17, further comprising, rendering at least one dynamically responsive graphical user interface element on the GUI to provide visual feedback to the user regarding the engagement state.

23. The method of claim 22, wherein the at least one dynamically responsive graphical user interface element changes in real time in response to a relationship between a movement of one of the hand controllers to a position of one of the robotic arms.

24. The method of claim 23, wherein a first portion of the at least one dynamically responsive graphical user interface element corresponds to a position and orientation of a first hand controller of the hand controllers, and a second portion of the at least one dynamically responsive graphical user interface element corresponds to a position and orientation of one of the robotic arms.

25. The method of claim 24, wherein a third portion of the at least one dynamically responsive graphical user interface element corresponds to a state of a tool coupled to one of the robotic arms.

26. The method of claim 17, further comprising rendering a representation of a first robotic arm of the robotic arms, and rendering a representation of a second robotic arm of the robotic arms.

27. The method of claim 25, further comprising, rendering on the GUI a representation of a grasper tool associated with one of the robotic arms.

28. The method of claim 25, further comprising, rendering on the GUI a representation of a scissor tool associated with one of the robotic arms.

29. The method of claim 16, wherein the visual representation of at least the engagement state between the hands of the user of the hand controllers includes a first set of two concentric rings and a second set of two concentric rings.

30. A non-transitory computer-readable medium storing computer-executable instructions stored therein, which when executed by at least one processor, cause the at least one processor to perform the operations of:
displaying on a display live video footage captured by the camera assembly,
overlaying the GUI on the live video footage on the display,
receiving by the processor input from one or more buttons or touch inputs on the hand controllers to control the robotic arm assembly,
rendering a graphical representation of the input on the GUI to provide the user with a visual representation of at least an engagement state between hands of the user and the hand controllers or a spatial awareness of the robotic arms within a cavity to control the surgical robotic system,
rendering on the GUI a graphical user interface element indicating a first mode of the surgical robotic system,
receiving by the processor a mode change indicator from the hand controllers, and responsive to the mode change indicator:
instructing the GUI to change the graphical user interface element indicating the first mode, and
causing the surgical robotic system to exit the first mode, and activate a second mode.

31. The non-transitory computer readable medium of claim 30, wherein the computer-executable instructions further cause the at least one processor to perform the operations of:
rendering on the GUI one or more selectable menu items associated with control of the robotic arms or one or more instruments coupled to the robotic arms.

32. The non-transitory computer readable medium of claim 31, wherein the robotic arm assembly is communicatively coupled to the hand controllers and are responsive to selection of one or more of the plurality of buttons or touch inputs to control the robotic arms or one or more instruments coupled to the robotic arms.

34. The non-transitory computer readable medium of claim 30, wherein the computer-executable instructions further cause the at least one processor to perform the operations of: rendering on the GUI one or more surgical robotic pose views to display a simulated view of the robotic arms or camera field of view of the camera assembly.

35. The non-transitory computer readable medium of claim 30, wherein a visual representation of the robotic pose views includes a view frustum.

36. The non-transitory computer readable medium of claim 31, wherein the computer-executable instructions further cause the at least one processor to perform the operations of:
rendering at least one dynamically responsive graphical user interface element on the GUI to provide visual feedback to the user regarding the engagement state.

37. The non-transitory computer readable medium of claim 36, wherein the at least one dynamically responsive graphical user interface element changes in real time in response to a relationship between a movement of one of the hand controllers to a position of one of the robotic arms.

38. The non-transitory computer readable medium of claim 37, wherein a first portion of the at least one dynamically responsive graphical user interface element corresponds to a position and orientation of a user's first hand on a first hand controller of the hand controllers, and a second portion of the at least one dynamically responsive graphical user interface element corresponds to a position and orientation of one of the robotic arms.

39. The non-transitory computer readable medium of claim 30, wherein the computer-executable instructions further cause the at least one processor to perform the operations of:
rendering a representation of a first arm of the robotic arms, and a representation of a second robotic arm of the robotic arms.

41. The non-transitory computer readable medium of claim 40, wherein the computer-executable instructions further cause the at least one processor to perform the operations of:
rendering on the GUI a representation of a grasper tool associated with one of the robotic arms.

42. The non-transitory computer readable medium of claim 41, wherein the computer-executable instructions further cause the at least one processor to perform the operations of: rendering on the GUI a representation of a scissor tool associated with one of the robotic arms.

43. The non-transitory computer readable medium of claim 42, wherein the visual representation of at least the engagement state between the hands of the user and the hand controllers includes a first set of two concentric rings and a second set of two concentric rings.
